(19) United States
(12) Patent Application Publication
(10) Pub. No.: US 2023/0398197 A1
(43) Pub. Date: Dec. 14, 2023

(54) COLDWATER DISEASE VACCINE COMPRISING AN ATTENUATED FLAVOBACTERIUM PSYCHROPHILUM STRAIN

(71) Applicant: University of Idaho, Moscow, ID (US)

(72) Inventor: Kenneth D. Cain, Moscow, ID (US)

(73) Assignee: University of Idaho, Moscow, ID (US)

(21) Appl. No.: 18/329,816

(22) Filed: Jun. 6, 2023

Related U.S. Application Data
(60) Provisional application No. 63/350,081, filed on Jun. 8, 2022.

(51) Int. Cl. A61K 39/02; A61P 37/04

(52) U.S. Cl. CPC A61K 39/0216; A61P 37/04; A61K 2039/552

(57) ABSTRACT

Bacterial cold-water disease (BCWD) vaccines for fish comprising, consisting of, or consisting essentially of, a live, attenuated Flavobacterium psychrophilum strain derived from a US149 strain and/or a US063 (ST278) strain, particularly formulated for fish of the Salmo and Salvelinus genera are disclosed. Disclosed embodiments also concern isolated, livem nonattenuated or attenuated Flavobacterium psychrophilum strains derived from an unattenuated strain having a cumulative percent mortality (CPM) in fish species in the Salmo and Salvelinus genera challenged with the strain of 40% or greater prior to attenuation. Treating BCWD comprises administering an effective amount of a disclosed vaccine to fish species primarily of the Salmo and Salvelinus genera. Making a BCWD vaccine comprises first identifying a highly virulent Flavobacterium psychrophilum bacterial strain for a target genus or species of interest, attenuating the highly virulent Flavobacterium psychrophilum bacterial strain, and then using the attenuated strain to produce a vaccine.

### CROSS REFERENCE TO RELATED APPLICATION

The present application claims the benefit of the earlier filing date of U.S. patent application Ser. No. 63/350,081, filed on Jun. 8, 2022, which is incorporated herein by reference. The present application is also related to subject matter disclosed by U.S. Pat. No. 7,740,864, and U.S. patent application Ser. Nos. 13/570,303 and 16/682,892, each of which is incorporated herein by reference in its entirety.

### FIELD

The present invention concerns fish vaccines, particularly a vaccine for bacterial coldwater disease (also referred to as rainbow trout fry syndrome) in fish, a method for administering the vaccine and a method for making the vaccine. A specific embodiment concerns a bacterial coldwater disease fish vaccine comprising an attenuated Flavobacterium psychrophilum strain, with one strain embodiment designated Fp063-np13, that is particularly formulated for administration to fish of the genera Salmo or Salvelinus, such as Atlantic salmon, that have proved resistant to protection using prior vaccines.

### BACKGROUND

Flavobacterium psychrophilum is a gram-negative bacterial fish pathogen that causes bacterial coldwater disease (BCWD). Flavobacterium psychrophilum is one of the most important pathogens affecting salmonid aquaculture due to its wide distribution and economic impact. Annual losses incurred from BCWD in the U.S. Pacific Northwest alone are approximately 9.6 and 4 million dollars for commercial aquaculture of rainbow trout (Oncorhynchus mykiss Walbaum) and conservation aquaculture of salmonid species, respectively. Preventative measures reduce risk factors, such as stress, poor water quality, and cutaneous lesions. But, even with these preventive measures in practice, BCWD commonly occurs and generally requires utilizing limited treatment options, such as reducing pathogen concentrations, eliminating the spread of the pathogen, and administering antibiotics. Treatment effectiveness can be inconsistent, and there are risks of bacterial strains developing resistance to the few approved antibiotics.

Rifampicin is a broad-spectrum antibiotic that inhibits bacterial DNA-dependent RNA polymerase. Rifampicin-resistant bacteria have been used to develop attenuated bacterial vaccines for diseases affecting fish, as disclosed by Klesius, U.S. Pat. Nos. 6,019,981; Shoemaker, 6,881,412 and 6,991,793; and Evans, 7,067,122. The attenuated live vaccines of Klesius, Shoemaker, and Evans were effective when administered to fish by immersion. This is not too surprising because fish are readily infected with enteric septicemia, columnaris disease, and Edwardsiella septicemia by immersion in water containing the causative organisms.

The same does not hold true for Flavobacterium psychrophilum and BCWD. Attenuated live vaccines for BCWD have been developed, including Flavobacterium psychrophilum B17-ILM attenuated vaccine (CSF259-93 as parent strain). However, initial vaccination trials utilizing the B17-ILM attenuated vaccine demonstrated limited or insufficient efficacy when tested in representative species in the genera Salmo or Salvelinus (i.e. Atlantic salmon Salmo salar and brook trout Salvelinus fontinalis).

Given the importance of these char species for commercial and resource-based hatchery production in North America and commercial Atlantic salmon production globally (both net-pen and land-based systems), an efficacious Flavobacterium psychrophilum BCWD vaccine must be developed for fish species of the Salmonidae family, such as the Salmo or Salvelinus genera, including by way of example Salmo salar (Atlantic salmon) and Salvelinus fontinalis (brook trout).

### SUMMARY

The B17-ILM attenuated vaccine has provided solid protection against a variety of Flavobacterium psychrophilum strains (Ma et al., 2018) when administered to rainbow trout (Oncorhynchus mykiss). The lack of protection in Atlantic salmon and limited protection in brook trout appear to be host related. Challenge trials have shown that the B17-ILM vaccine parent strain CSF259-93 is essentially avirulent in Atlantic salmon and showed limited virulence in brook trout. Therefore, it is likely that the attenuated B17-ILM vaccine strain does not adequately enter the host and elicit immunity in these species when compared to rainbow trout. This is further supported by the fact that in rainbow trout the B17-ILM vaccine provides cross protection against some of the same strains used in the trials with Atlantic salmon and brook trout. For example, the US149 strain (sequence type 70 -ST70), originally isolated from Atlantic salmon in Washington, USA, was virulent in all three species but only vaccinated rainbow trout were protected when challenged with this isolate (Ma et al., 2018).

The present inventors have determined that new and effective attenuated BCWD vaccines should be derived from highly virulent bacterial strains in the target species (or genus) of interest. This is the case, for example, for CSF259-93 (ST10) in rainbow trout. Bruce et al. (2021) determined that a US149 strain and a US063 strain (isolated from lake trout Salvelinus namaycush, in Michigan, USA) cause high cumulative percent mortality (CPM) relative to CSF259-93 in Atlantic salmon, CPM 97% and 81% respectively, while challenge with the CSF259-93 only resulted in 1.3% CPM. In brook trout the US149 and the US063 strains resulted in CPM of 40% and 73% respectively, while CPM from the CSF259-93 strain was only 18.7%. The present inventors therefore conceived of creating a completely attenuated, rifampicin-resistant F. psychrophilum strain derived from highly virulent bacterial strains, as exemplified by the US149 and US063 (ST278) strains, to provide protection for fish species in Salmonidae family.

Accordingly, certain disclosed embodiments of the present invention concern a bacterial cold-water disease (BCWD) vaccine for fish in the Salmonidae family comprising, consisting of, or consisting essentially of, an attenuated Flavobacterium psychrophilum strain. The attentuated strain is prepared from an tinattenuated Flavobacterium psychrophilum parent strain having a 40% or greater cumulative percent mortality (CPM) in fish weighing from 5-10 grams that are challenged intramuscularly with the parent strain at a dose of at least 1×10⁷ cfu/fish. For certain embodiments, the unattenuated. Flavobacterium psychrophilum parent strain has a 50%, a 60%, a 70% or greater cumulative percent mortality (CPM) in fish weighing from 5-10 grams that are challenged intramuscularly with the parent strain at a dose of at least 1×10⁷ cfu/fish. For example, where the unattenuated Flavobacterium psychrophilum parent strain was US063. the CPM in Atlantic salmon was 50% or greater and the CPM in brook trout was 60% or greater. And where the unattenuated Flavobacterium psychrophilum parent strain was US149, the CPM in Atlantic salmon was 90% or greater and the CPM in brook trout was 40% or greater. Accordingly. certain vaccine embodiments comprise a live attenuated Flavobacterium psychrophilum strain derived from US149, US063 (ST278), or both. A person of ordinary skill in the art will appreciate that the vaccine can comprise two or more different live, attenuated Flavobacterium psychrophilum strains. Furthermore, certain disclosed vaccine embodiments are particularly formulated for Atlantic salmon, are particularly formulated for fish in the Salmonidae family, and/or are formulated for all species of Salmo or Salvelinus genera, such as Salmo salar (Atlantic salmon) or Salvelinus fontinalis (brook trout).

A currently preferred Flavobacterium psychrophilum strain is designated Fp063-np13. The Fp063-np13 strain was deposited with the United States Department of Agriculture, Agriculture Research Culture Collection (also known as the Northern Regional Research Laboratory, or NRRL) on Apr. 27, 2022, and was assigned deposit number B-68156.

Disclosed vaccine embodiments typically comprise 10 3 CFU/ml to 10 10 CFU/ml of the live attenuated Flavobacterium psychrophilum, and more typically comprise 10 6 CFU/ml to 10 9 CFU/ml of the live attenuated Flavobacterium psychrophilum. A person of ordinary skill in the art will also appreciate that disclosed vaccines may comprise any additional agent typically used for formulating vaccine compositions, such as water, physiological saline, an oil, such as mineral oil or a vegetable oil, aqueous sodium carboxymethyl cellulose, aqueous polyvinylpyrrolidone, alum, an adjuvant, such as Freund's incomplete adjuvant, or combinations thereof. The Flavobacterium psychrophilum may be partially attenuated, but is preferably completely attenuated, such as is provided by the Flavobacterium psychrophilum Fp063-np13 strain.

The present invention also concerns an isolated, attenuated Flavobacterium psychrophilitin prepared from an unatteritiated Flavobacterium psychrophilum parent strain having a 40% or greater cumulative percent mortality (CPM) in fish species in the Salmonidae family weighing from 5-10 grams that are challenged intramuscularly with the parent strain at a dose of at least 1×10⁷ cfu/fish. Again, a particularly suitable Flavobacterium psychrophilum strain is designated Fp063-np13. Such isolated, attenuated Flavobacterium psychrophillini strains typically have a protein or carbohydrate profile that is different from a corresponding non-attenuated Flavobacterium psychrophilum.

Certain disclosed embodiments concern a method for treating fish comprising providing a disclosed BCWD vaccine comprising, consisting of, or consisting essentially of, a live attenuated Flavobacterium psychrophilum strain, and administering an effective amount of the vaccine to the fish species, such as fish species of Salmonidae family, such as the Salmo and Salvelinus genera. The method may comprise administering the vaccine by any suitable method, such as immersion in a dip or bath, or by injection. The vaccine may be administered by immersion, such as by immersing fish in a 1:10 to 1:10,000 volume aqueous dilution of an initial vaccine composition comprising 10⁸-10¹⁰ cfu of a suitable Flavobacterium psychrophilum strain/ml, depending on the minimum protective dose. Fish are immersed for an effective period of time, with certain embodiments immersing for 1 to 30 minutes. A standard dose for injection administration to individual fish is from 10⁴ CFU/fish to 10⁸ CFU/fish of the live, attenuated Flavobacterium psychrophilum. The method may comprise administering a booster to previously vaccinated fish, such as administering a booster two to four weeks after initial vaccination. The method also may comprise administering a second therapeutic to the fish species, such as an antibiotic, including oxytetracycline or florfenicol.

A method for making a CWD vaccine also is disclosed. One embodiment of the method comprises first identifying a highly virulent Flavobacterium psychrophilum bacterial strain for a target genus or species of interest. This highly virulent Flavobacterium psychrophilum bacterial strain is then attenuated and used to produce a live, attenuated vaccine comprising, consisting of, or consisting essentially of, the attenuated Flavobacterium psychrophilum bacterial strain. The highly virulent attenuated Flavobacterium psychrophilum bacterial strain is prepared from an unattenuated Flavobacterium psychrophilum parent strain having a 40% or greater cumulative percent mortality (CPM) in fish weighing from 5-10 grams that are challenged intramuscularly with the parent strain at a dose of at least 1×10⁷ cfu/fish. Attenuation can be accomplished by serial passaging in media comprising increasing concentrations of rifampicin, such as rifampicin concentrations in tryptone yeast extract salt (TYES) ranging from greater than On rifampicin/ml media to at least 320 μg rifampicin/ml media, although attenuation likely can be achieved at lower concentrations of about 260 μg rifampicin/ml media. The method may further comprise growing live, attenuated highly virulent Flavobacterium psychrophilum bacterial strain or strains on iron-limited media.

The foregoing and other objects, features, and advantages of the invention will become more apparent from the following detailed description, which proceeds with reference to the accompanying figures.

### BRIEF DESCRIPTION OF THE DRAWINGS

FIG. 1 is graph of cumulative percent mortality versus days post challenge for Atlantic salmon vaccinated with B.17ILM vaccine followed by a challenge trials with the listed strains for Trial A.

FIG. 2 is a bar graph of cumulative percent mortality versus days post challenge for fish vaccinated with B.17ILM vaccine followed by challenge trials with the listed strains for the data presented by FIG. 1.

FIG. 3 is graph of cumulative percent mortality versus days post challenge for Atlantic salmon vaccinated with B.17ILM vaccine followed by a challenge trials with the listed strains for Trial B.

FIG. 4 is a bar graph of cumulative percent mortality versus days post challenge for fish vaccinated with B.17ILM vaccine followed by challenge trials with the listed strains for the data presented by FIG. 3.

FIG. 5 is graph of cumulative percent mortality versus days post challenge for brook trout vaccinated with B.17ILM vaccine followed by a challenge trials with the listed strains.

FIG. 6 is a bar graph of cumulative percent mortality versus days post challenge for fish vaccinated with B.17ILM vaccine followed by challenge trials with the listed strains for the data presented by FIG. 5.

FIG. 7 is a schematic illustrating attenuation of Flavobacterium psychrophilum strains by serial passaging on TYES agar comprising increasing concentrations of rifampicin.

FIG. 8 shows rifampicin-resistant colonies with different passages on TYES plates with increasing concentrations of rifampicin: A-C, US063 selected strain; D-F, US149 selected strain; A and D, the initial TYES selected plates with 2μg/ml rifampicin; B and E, the TYES plates with 20 μg/ml rifampicin; and C and F, the TYES plates with 260 μg/ml rifampicin.

FIG. 9 is a graph of Optical Density (OD) at 525 nanometers versus time showing growth curves of the Flavobacterium psychrophilum parent US063 and rifampicin-resistant strain Fp063-np13, where the error bars indicate the standard error of the mean.

FIG. 10 is an image of an SDS-PAGE (sodium dodecyl sulfate-polyacrylamide gel electrophoresis where: STD provides pre-stained protein standards of known molecular weight (kilodaltons); lane 1 is an attenuated 063 strain grown on an iron-limited medium; lane 2 is an 063 strain attenuated on TYES medium; lane 3 is US063 parent strain; lane 4 is an attenuated Flavobacterium psychrophilum for the known B.17 vaccine; and lane 5 is Flavobacterium psychrophilum strain CSF259-93.

FIG. 11 is Western blot made using Atlantic salmon immune serum for identifying immunogenic regions of CSF 259-93 and US063 in comparison to the attenuated vaccine strain, where: STD provides pre-stained protein standards of known molecular weight (kilodaltons); lane 1 is an attenuated 063 strain grown on an iron-limited medium; lane 2 is an attenuated 063 strain grown on TYES medium; lane 3 is US063 parent strain; lane 4 is an attenuated Flavobacterium psychrophilum for the known B.17 vaccine; and lane 5 is B.17 Flavobacterium psychrophilum parent strain CSF259-93.

FIG. 12 is Western blot made using Rainbow trout immune serum for identifying immunogenic regions of CSF 259-93 and US063 in comparison to the attenuated vaccine strain, where: STD provides pre-stained protein standards of known molecular weight (kilodaltons); lane 1 is an attenuated 063 strain grown on an iron-limited medium; lane 2 is an attenuated 063 strain grown on TYES medium; lane 3 is US063 parent strain; lane 4 is an attenuated Flavobacterium psychrophilum for the known B.17 vaccine; and lane 5 is B.17 Flavobacterium psychrophilum parent strain CSF259-93

FIG. 13 is a graph of strains versus cumulative percent mortality (CPM) in Atlantic salmon 28 days after intramuscular injection challenge with data presented as mean±SE, where different letters indicate significant difference (p<0.05) and shared or same letters indicate no significant difference between treatments.

FIGS. 14A and 14B are photographs showing clinical signs in Atlantic salmon after intramuscular challenge with US149 (FIG. 4A) or US063 (FIG. 4B).

FIG. 15 is graph of strains versus cumulative percent mortality (CPM) in Atlantic salmon 28 days after intraperitoneal injection challenge with different strains of Flavobacterium psychrophilum, where the data is presented as mean±SE, and ** indicates statistically significant difference (p<0.01).

FIGS. 16A and 16B are photographs showing clinical signs in Atlantic salmon after intraperitoneal challenge with US149 (FIG. 6A) or US063 (FIG. 6B).

FIG. 17 is a graph of species versus ELISA titer showing specific anti-F. psychrophilum immune response in Atlantic salmon 28 days after intramuscular challenge with parent or rifampicin-resistant strains, where the ELISA value is the average titer of five individual fish from each treatment group, except for US149 as only two fish survived, following challenge, and the data is presented as mean±SE, with ** indicating a statistically significant difference (p<0.01).

FIG. 18 is a graph of species versus ELISA titer showing specific anti-F. psychrophilum immune response in Atlantic salmon 28 days or 14 days after intraperitoneal challenge with parent or rifampicin resistant strains, where the ELISA value is the average titer of five individual fish from each treatment group.

FIG. 19 is a chart showing specific anti-F. psychrophilum immune response in vaccinated and non-vaccinated Atlantic salmon 28 days after IM challenge with virulence strains, where the ELISA value is an average titer of five individual fish from each treatment group.

FIG. 20 is a graph of CPM (%) versus time (days post challenge) showing cumulative percent mortality among IP-vaccinated and non-vaccinated Atlantic salmon after IM challenge with F. psychrophilum virulent strains.

FIG. 21 is a graph of CPM (% after 28 days) versus strain showing cumulative percent mortality among vaccinated (Vac) and control (Ctl) Atlantic salmon after IM challenge with F. psychrophilum US063 virulent strain.

FIGS. 22A-22G are graphs of ELISA titer versus treatment groups showing that vaccinated fish had significantly higher F. psychrophilum antibody titers than non-vaccinated from 6 weeks post vaccination, and at 14 weeks post vaccination, the 1/10 dilution group had the highest titer, but without significant difference with other dilution groups.

FIG. 23 is a graph showing cumulative percent mortality (CPM, % after 28 days) for vaccinated (Vac) and control (Ctl) rainbow trout after IM challenge with F. psychrophilum virulent strains.

FIG. 24 is a graph of ELISA titer versus time (weeks) for vaccinated (Vac) and control (Ctl) rainbow trout showing specific anti-F. psychrophilum immune response in rainbow trout post immersion vaccination using a Fp063-np13 live attenuated Flavobacterium psychrophilum vaccine.

FIG. 25 is a graph showing cumulative percent mortality (CPM, % after 21 days) for non-vaccinated control (NonVac CTL), Fp063-np13-TYES immersion vaccinated rainbow trout, and Fp063-np13-ILM immersion vaccinated rainbow trout after challenge with 50 μl of a virulent F. psychrophilum US063 strain at 10 weeks post initial immunization.

FIGS. 26A-26C provide graphs of ELISA titer 4 weeks, 6 weeks, and 8 weeks post-vaccination for each of the treatment groups tested, as discussed by Example 4.

### DETAILED DESCRIPTION

### I. Abbreviations, Terms and Definitions

A. Abbreviations
CFU: Colony forming units.
CPM: Cumulative percent mortality.
BCWD: Bacterial Coldwater Disease.
DPI: Days post injection.
ELISA: Enzyme-linked immunosorbent assay.
IP: Intraperitoneal.
OD: Optical density.
RPS: Relative percent survival.
TYES: tryptone yeast extract salt.
B. Terms and Definitions

Unless otherwise noted, technical terms are used according to conventional usage as would be understood by a person of ordinary skill in the art. Definitions of common terms in molecular biology may be found in Lewin's Genes X, ed. Krebs et al, Jones and Bartlett Publishers, 2009 (ISBN 0763766321); Kendrew et al. (eds.), The Encyclopedia of Molecular Biology, Blackwell Publishers, 1994 (ISBN 0632021829); Robert A. Meyers (ed.), Molecular Biology and Biotechnology: A Comprehensive Desk Reference, Wiley, John & Sons, Inc., 1995 (ISBN 0471186341); and George P. Rédei, Encyclopedic Dictionary of Genetics, Genomics, Proteomics and Informatics, 3rd Edition, Springer, 2008 (ISBN: 1402067534).

The following explanations of terms and abbreviations are provided to better describe the present disclosure and to guide those of ordinary skill in the art to practice the present disclosure.

As used herein, “comprising” means “including.”

The singular forms “a” or “an” or “the” refer to one or more than one unless the context clearly dictates otherwise.

The term “or” refers to a single element of stated alternative elements or a combination of two or more elements, unless the context clearly indicates otherwise. All publications, patent applications, patents, and other references mentioned herein are incorporated by reference in their entirety for all purposes.

Although methods and materials similar or equivalent to those described herein can be used to practice or test the present disclosure, suitable methods and materials are described below. The materials, methods, and examples are illustrative only and not intended to be limiting. Other features of the disclosure will be apparent to a person of ordinary skill in the art from the following detailed description and the claims.

Unless otherwise indicated, all numbers expressing quantities of components, molecular weights, percentages, temperatures, times, and so forth, as used in the specification or claims are to be understood as being modified by the term “about.” Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximates unless the word “about” is recited.

Adjuvant: The term “adjuvant” as used herein means any substance or vehicle that enhances the effectiveness of a disclosed immunogenic composition, such as by enhancing the immune response to an antigen (for example an CWD antigen) by an animal's immune system. An adjuvant can be used to form a composition or compositions disclosed herein, for example as part of a CWD vaccine composition. Adjuvants included in some embodiments of a composition disclosed herein can include, but are not limited to, sorbitan oleate, sorbitol-T, aluminum salts, such as aluminum phosphate or aluminum hydroxide; various types of oils, such as vegetable oil and mineral oil, Freund's complete adjuvant; Freund's incomplete adjuvant; Carbomer-based adjuvants, such as those containing 934P or 971P; polymer-based adjuvants, such as Carbigen™ or Polygen™; immune-stimulating complexes (ISCOMs); liposomes; polysaccharides; derivatized polysaccharides; oligonucleotides; cytokines; bacterial derivatives; viral derivatives; or combinations thereof.

Administer, Administering, Administration: As used herein, administering a composition (e.g. an immunogenic composition) means to apply, give, or bring the composition into contact with an animal, particularly fish. Administration can be accomplished by a variety of routes, such as, for example, topical, oral, subcutaneous, transdermal, immersion, intrathecal, intramuscular, intravenous, intraperitoneal, intranasal, and similar routes, or combinations thereof, with immersion being a particularly preferred method of administration.

Ameliorating: Refers to a reduction in the number or severity of one or more signs or symptoms of a disease.

Antibody: An “antibody” is an immunoglobulin molecule evoked in animals by a specific antigen (immunogen). Antibodies are characterized by reacting specifically with the antigen in some demonstrable way. “Eliciting an antibody response” refers to the ability of an antigen or other molecule to induce the production of antibodies.

Antigen: “Antigen” refers to a compound, composition, or substance that can stimulate the production of antibodies or a T-cell response in an animal, including compositions that are injected or absorbed into an animal.

Attenuated, Attenuation: An “attenuated” bacterium is weakened and/or less virulent as compared to a non-attenuated form capable of causing disease. Attenuated bacterium may stimulate an immune response and/or immunity but are not capable of causing disease. Attenuated bacterium may be used to stimulate an immune response and/or induce immunity in a recipient, such as fish.

Combination: A combination includes two or more components that are administered such that the effective time period of at least one component overlaps with the effective time period of at least one other component. A component may be a composition. In some embodiments, the effective time periods of all components administered overlap with each other. In an exemplary embodiment of a combination comprising three components, the effective time period of the first component administered may overlap with the effective time periods of the second and third components, but the effective time period of the second component independently may or may not overlap with that of the third component. In an exemplary embodiment of a combination comprising four components, the effective time period of the first component administered overlaps with the effective time periods of the second, third, and fourth components; the effective time period of the second component overlaps with those of the first and fourth components, but not that of the third component; and the effective time period of the fourth component overlaps with that of the second and third components only. A combination may be a composition comprising the components, a composition comprising two or more individual components, or a composition comprising one or more components and another separate component (or components) or composition(s) comprising the remaining component(s). In some embodiments, the two or more components may comprise two or more different components administered substantially simultaneously or sequentially in any order, the same component administered at two or more different times, or a combination thereof.

Completely Attenuated: Refers to bacteria that have no capability of producing disease.

Does not Effectively Cause Disease: Means, in reference to the immersion of fish in water containing an infectious bacterium, that less than 10% of fish immersed for 30 minutes in water at 15° C. containing 1×10 CFU (colony forming units) per ml of the bacterium will become infected with the bacterium and manifest signs of disease due to infection with the bacterium.

Effective amount: The term “effective amount” refers to an amount of an agent (such as one or more compositions disclosed herein either alone, or in combination with other therapeutic agent(s)) that is sufficient to induce a desired biological result. That result may be amelioration or alleviation of the signs, symptoms, or causes of a disease, or any other desired alteration of a biological system. The amount can vary with the condition being treated, the stage of advancement of the condition, and the type and concentration of formulation applied. In some embodiments, an effective amount is an amount which, when administered to a subject, is sufficient to engender a detectable immune response. Such a response may comprise, for instance, generation of an antibody. Appropriate amounts will be readily apparent to those of ordinary skill in the art or can be determined by routine experimentation based on information provided herein, such as vaccination followed by a challenge wherein the vaccinated animal performs better than a non-vaccinated animal that is challenged similarly.

Immune response: An “immune response” is a response of a cell of the immune system, such as a B-cell, T-cell, macrophage or polymorphonucleocyte, to a stimulus, such as an antigenic peptide. An immune response can include any cell of the body involved in a host defense response, including for example, an epithelial cell that secretes an interferon or a cytokine. An immune response includes, but is not limited to, an innate immune response or inflammation. As used herein, a protective immune response refers to an immune response that protects a subject from infection (prevents infection or prevents the development of disease associated with infection).

Immune stimulatory composition: The terms, “immune stimulatory composition” and “immunogenic composition” used herein mean a composition useful for stimulating or eliciting an immune response (or immunogenic response) in a subject. Disclosed immune stimulatory compositions can include an attenuated live bacterium.

Inactivated: In the context of the present disclosure, an “inactivated” bacterium is one that is not capable, or has substantially reduced capability of, establishing an infection in a host or host cell. Bacterium can be inactivated using, for example, chemicals, such as antibiotics like rifampicin, heat, alterations in pH and/or irradiation (such as ultraviolet or gamma irradiation).

Infection: Infection or challenge means that a subject has been exposed to organisms, such as bacteria, that may produce disease causing the subject to suffer one or more clinical signs of the diseases when they have been exposed to such organisms.

Mass Vaccination: Refers to methods of vaccination that do not require handling individual fish. Methods of mass vaccination include oral, spray, and immersion administration.

Preventing: Preventing a disease refers to inhibiting the full development of a disease.

Treating: Refers to a therapeutic intervention, such as administration of a vaccine comprising a live, attenuated bacteria, such as attenuated Flavobacterium psychrophilum, that ameliorates a sign or symptom of a disease or pathological condition prior to or after it has begun to develop.

Purified: The term “purified” does not require absolute purity; rather, it is intended as a relative term. Thus, for example, a purified protein, bacterium, nucleic acid, or other compound is one that is isolated in whole or in part from associated proteins and other contaminants. In certain embodiments, the term “substantially purified” refers to a protein, bacterium, nucleic acid, or other compound that has been isolated from a cell, cell culture medium, or other crude preparation and subjected to purification to remove various components of the initial preparation, such as proteins, cellular debris, and other components.

Subject: A “subject” is any multi-cellular vertebrate organism, with certain disclosed embodiments of the present invention particularly concerning aquatic species, such as fish, particularly fish in the Salmo and Salvelinus genera.

Vaccine: “Vaccine” refers to an immunogenic material, or a composition comprising an immunogenic material, capable of stimulating an immune response. Vaccines may be administered to prevent, ameliorate, or treat an infectious or other type of disease or diseases. The immunogenic material may include attenuated or inactivated microorganisms (such as bacteria or viruses), or antigenic proteins (including VLPs), peptides, or DNA derived from or encoding them, or combinations thereof. An attenuated vaccine is a virulent organism that has been modified to produce a less virulent form, but nevertheless retains the ability to elicit antibodies and an immune response against the virulent form. Vaccines may be administered with an adjuvant to boost the immune response.

Wounded: Having a non-physiologic portal of entry to bacteria in water. Examples of wounds include lacerations, punctures, and traumatic removal of one or more fins or scales.

### II. Introduction

Flavobacterium psychrophilum B17-ILM attenuated vaccine has provided solid protection to a variety of strains (Ma et al., 2018) when administered to rainbow trout (Oncorhynchus mykiss). Surprisingly, vaccination trials utilizing Flavobacterium psychrophilum B17-ILM attenuated vaccine (CSF259-93 as parent strain) have demonstrated no or limited efficacy when tested in representative species in the genera Salmo or Salvelinus (i.e. Atlantic salmon Salmo salar and brook trout Salvelinus fontinalis).

Vaccination and challenge trials were conducted using a number of challenge strains, as indicated below in Table 1.

TABLE 1

| STRAIN | HOST | GEOGRAPHIC | SEQUENCE |
|---|---|---|---|
| ID | SPECIES | ORIGIN | TYPE |
| 03-179 | Steelhead trout | WA, USA | ST294 |
| 622-97 | Atlantic salmon | Chile | ST79 |
| US063 | Lake trout | MI, USA | ST278 |
| US149 | Atlantic salmon | WA, USA | ST70 |
| US157 | Atlantic salmon | OR, USA | ST11 |
| *CSF 259-93 | Rainbow trout | ID, USA | ST10 |

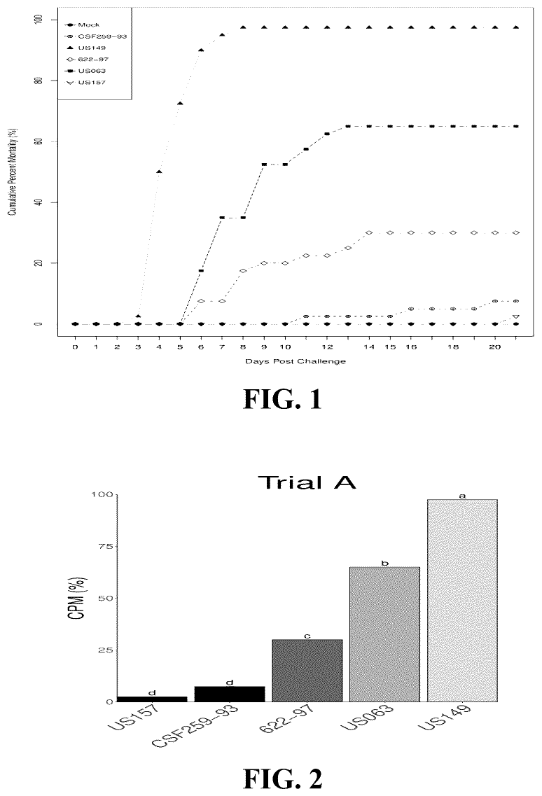

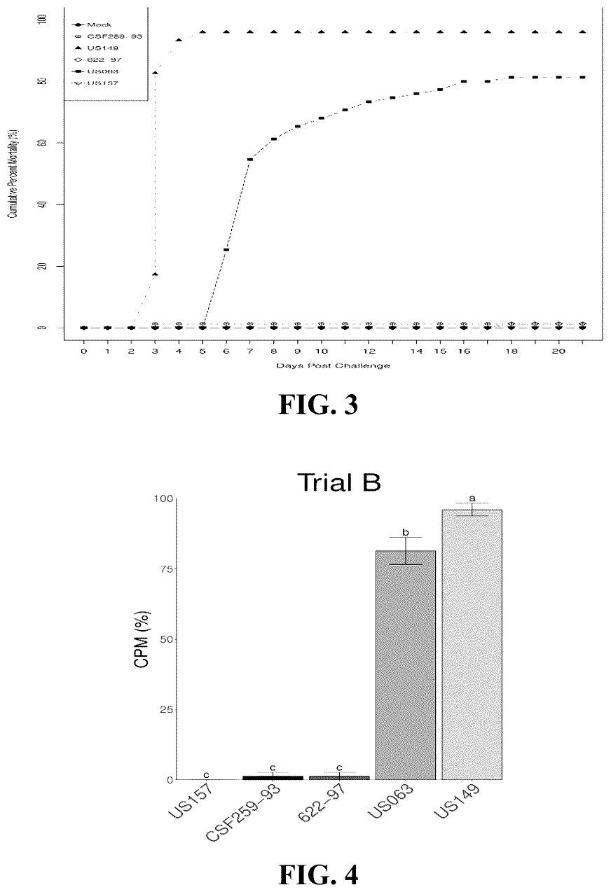

Specifically, Atlantic salmon and brook trout were vaccinated using the B.17ILM vaccine, and then vaccinated fish were challenged with the strains indicated above. The results of these trials are provided by FIGS. 1-6 and are summarized below by Tables 2 and 3.

TABLE 2
Results for Immersion Vaccination of Atlantic
Salmon Followed by Challenge Trials

| | | |
|---|---|---|
| CHALLENGE | TRIAL 1 | TRIAL 2 |

| STRAIN | CPM | RPS | CPM | RPS |
|---|---|---|---|---|
| 622-97V | 0 | 100 | 29.3 | — |
| 62297-C | 1.3 | | 21.3 | |
| 157-V | 2.7 | — | 0 | 0 |
| 157-C | 0 | | 0 | |
| 149-V | 96 | 0 | 81.3 | — |
| 149-C | 96 | | 78.7 | |
| 063-V | 84 | — | 86.7 | — |
| 063-C | 81.3 | | 78 | |
| 259-V | 1.3 | 0 | 2.7 | 0 |
| 259-C | 1.3 | | 2.7 | |

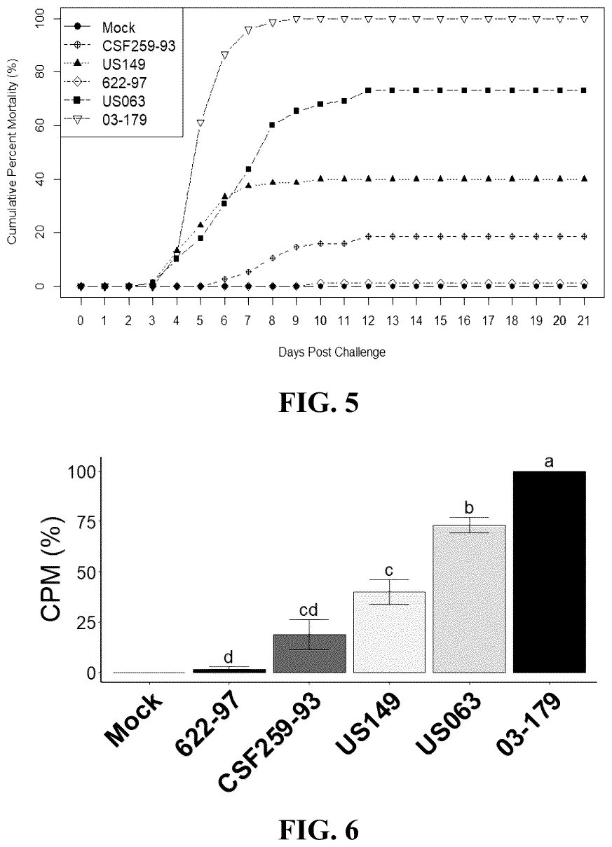

The information provided by FIGS. 1-4 and Table 2 establish that the B.17ILM vaccine did not provide protection in Atlantic salmon challenged with the listed F. psychrophilum strains. Similarly, FIGS. 5-6 and Table 3 establish that the B.17ILM vaccine did not provide significant protection in brook trout challenged with the listed F. psychrophilum strains.

TABLE 3
Results for Immersion Vaccination of Brook
Trout Followed by Challenge Trials

| CHALLENGE STRAIN | CPM | RPS |
|---|---|---|
| 622-97V | 0 | 100.0 |
| 622-97C | 1.3 | |
| 149-V | 37.3 | 6.8 |
| 149-C | 40 | |
| 03179-V | 96 | 4.0 |
| 03179-C | 100 | |
| 063-V | 70.7 | 3.3 |
| 063-C | 73.1 | |
| 259-V | 12 | 35.8 |
| 259-C | 18.7 | |

Based on these vaccination and challenge trials, it was clear that a new vaccine had to be developed at least for the protection of Salmo and Salvelinus genera, including Atlantic salmon and brook trout. Genetic diversity across strains has been well documented (see, for example,

Knupp et al., 2019). The present inventors postulated that the lack of protection in Atlantic salmon and limited protection in brook trout may be host related. Challenge trials established that the B17-ILM attenuated vaccine parent strain CSF259-93 is essentially avirulent in Atlantic salmon and showed limited virulence in brook trout. Therefore, it appears that the attenuated B.17 ILM vaccine strain does not adequately enter and elicit immunity in these species when compared to rainbow trout. This is further supported by the fact that in rainbow trout the B.17 ILM vaccine provides cross protection against some of the same strains used in the trials with Atlantic salmon and brook trout. For example, the US149 strain (sequence type 70 -ST70), originally isolated from Atlantic salmon in Washington, USA, was virulent in all three species but only vaccinated rainbow trout were protected when challenged with this isolate (Ma et al., 2018).

To address the deficiencies of the Flavobacterium psychrophilum B17-ILM attenuated vaccine and its inability to confer protection against CWD to, for example, Atlantic salmon and brook trout, certain disclosed embodiments concern producing an effective attenuated vaccine from bacterial strains that show high virulence in the target species (or genus). Bruce et al. (2021) showed that the US149 strain and the US063 strain (isolated from lake trout Salvelinus namaycush, in Michigan, USA) cause high cumulative percent mortality (CPM) relative to CSF259-93 in Atlantic salmon, with CPM values of 97% and 81% respectively. Challenge with CSF259-93 resulted in only 1.3% CPM. In brook trout the US149 and the US063 strains resulted in CPM values of 40% and 73% respectively, while CPM from the CSF259-93 strain was only 18.7%.

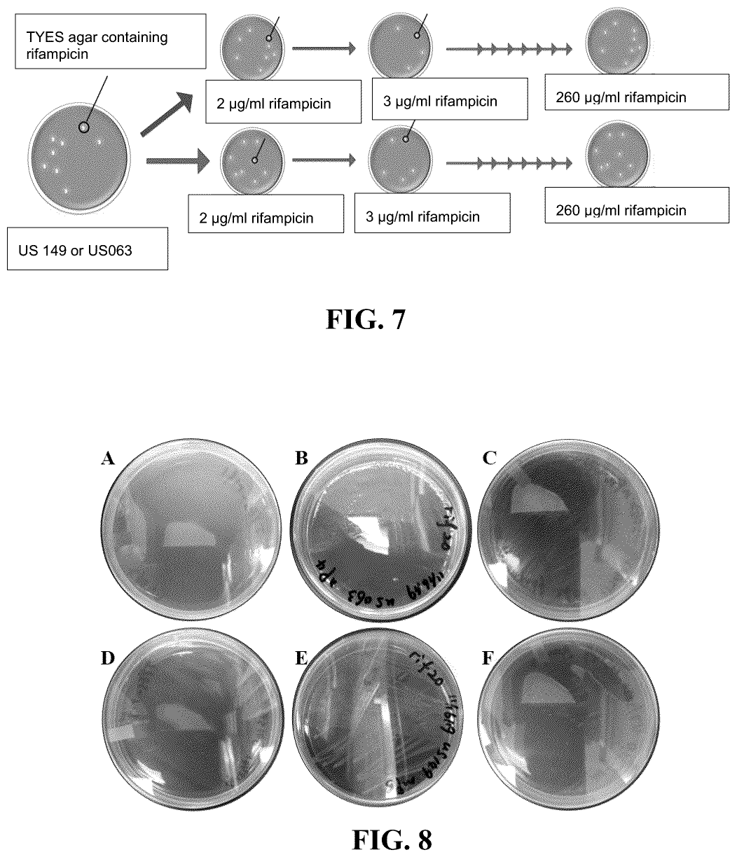

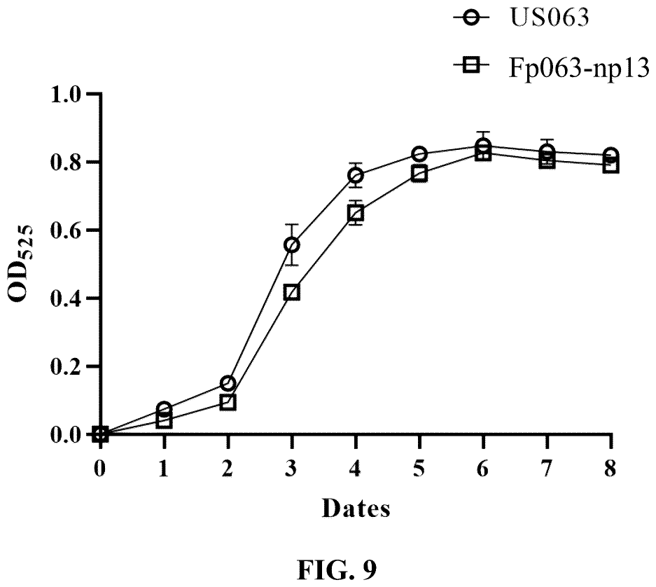

Accordingly, a rifampicin-resistance strategy was applied to Flavobacterium psychrophilum strains US149 and US063 to generate six selected strains (3 from each isolate) by serial passage on TYES plates containing increasing rifampicin concentrations, as illustrated schematically in FIG. 7. Complete attenuation was demonstrated for certain initially highly virulent strains, such as the Fp063-np13 strain, following bacterial challenges in Atlantic salmon. As a result, the present disclosure concerns isolated, attenuated, rifampicin-resistant Flavobacterium psychrophilum strains produced using the US149 strain, the US063 (ST278) strain, or both. The present disclosure also concerns an efficacious CWD vaccine comprising, consisting of, or consisting essentially of, a live, initially highly virulent but attenuated Flavobacterium psychrophilum strain, or strains, particularly suitable for treating North America and commercial Atlantic salmon and other species within the Salmo and Salvelinus genera. Injection vaccination elicited Flavobacterium psychrophilum-specific antibodies at 4 weeks post vaccination and high levels of protection in Atlantic salmon following challenge with virulent US063 or US149 strains.

### III. Description of Disclosed Embodiments

Certain disclosed embodiments concern a vaccine for protecting fish against a bacterial disease. The vaccine comprises, consists of, or consists essentially of, an attenuated live bacteria, with or without culture medium in which the bacteria were grown, that provides protection against disease caused by non-attenuated live bacteria of the same species as the attenuated bacteria. If desired, the vaccine may comprise constituents in addition to the attenuated bacteria, such as a carrier or vehicle. Suitable exemplary carriers include water, physiological saline, mineral oil, vegetable oil, aqueous sodium carboxymethyl cellulose, and aqueous polyvinylpyrrolidone. Vaccine formulations may also comprise adjuvants, or other pharmaceutically active agents. Suitable adjuvants include, but are not limited to, mineral oil, vegetable oil, alum, and Freund's incomplete adjuvant.

A preferred attenuated live bacteria is a strain that has been derived from Flavobacterium psychrophilum. Preferably, the attenuated Flavobacterium psychrophilum is non-virulent, and completely attenuated. Preferably, the attenuated Flavobacterium psychrophilum has a protein expression profile that differs from that of its parent pathogenic strain. Suitable attenuated Flavobacterium psychrophilum strains within the scope of the present claims can be the exact strains, or strains derived directly from, US149, US063, and/or Fp063-np13 strains. Strains US149 and US063 are deposited with the American Type Culture Collection (ATCC) in Manassas, Virginia. The Fp063-np13 strain has been deposited with the NRRL on Apr. 27, 2022, as stated above. Disclosed vaccine compositions may comprise a combination of attenuated live bacteria isolates, such as two or more strains developed from strains designated as US149 and US063, such as Flavobacterium psychrophilum strain designated Fp063-np13 and at least one additional strain. Flavobacterium psychrophilum strains within the scope of the claims can be, but need not be, derived directly from US149 and US063, or can include particular designated strains, such as Fp063-np13, or strains that are derived therefrom. Nor do the claims require access to particular designated strains, such as Fp063-np13. Instead, infringing Flavobacterium psychrophilum strains will have physical or chemical characteristics substantially similar to or identical to strains derived directly from US149 and US063, such as physical or chemical characteristics substantially similar to or identical to those of the Fp063-np13 strain, that allow production of fish vaccines effective for treating fish that are not now effectively treated with known vaccines, such as the B17-ILM attenuated vaccine.

Additional embodiments concern treating fish with vaccine embodiments within the scope of the present invention. For example, according to one disclosed embodiment, fish are mass vaccinated with an amount of attenuated live bacteria effective to provide protection against disease caused by non-attenuated live bacteria of the same species as the attenuated bacteria. In one embodiment, mass vaccination is by immersion.

Fish that may be treated by the method include any fish that is susceptible to infection and disease caused by the particular organism. The fish may be a fresh-water, marine or salt-water fish. Examples of fish that can be treated with disclosed vaccine embodiments include salmonids (Oncorhynchus sp., Salmo sp., and Salvelinus sp.), American, European, and Japanese eels (Anguilla sp.), tilapia (Oreochromis sp.), striped bass and hybrid-striped bass (Morone chrysops. and M. Saxatilis), flounders (Seriola sp.), Seabream (Sparus sp.), sea perch (Lates calcarifer), the estuarine grouper (Epinephelus tawine), walleye (StitzOste dion vitreum), channel catfish (Ictalurus punctutus), centra chids (such as largemouth bass, Micropterus salmoides), brown bullheads (Nebulosus sp.), fat head minnows (Pime phales promelas), golden shiners (Netenigonus crysoleu cas), goldfish (Carassius auratus), carp (Cyprinus Carpio), and aquarium fish species such as black mollies (Poecilia sphenops) and platies (Xiphosphorus maculatus).

Disclosed embodiments are particularly suitable for addressing CWD. Species affected specifically by CWD include all salmonids. The pathogen has also been reported in non-salmonid species, such as eel (Anguilla sp.), sea lamprey (Petromyzon marinus), carp (Cyprinus carpio), tench (Tinca tinca), crucian carp (Carassius carassius), goldfish (Carassius auratus), ayu (Plecoglossus altivelis), pale chub (Zacco platypus), European perch (Perca fluviatilis), roach (Rutilus rutilus), and others.

The present invention is particularly suitable for treating fish, such as for BCWD, that are not effectively treated with prior known vaccines, such as the B.17 ILM vaccine. While the B.17 ILM vaccine has proved quite effective, it has not proved effective for treating all fish species. Accordingly, the present invention is particularly directed to vaccines for all species and subspecies within the Salmonidae/Salmoninae family, as disclosed by https://www.fishbase.in/Summary/FamilySummary.php?ID=76, which information is incorporated herein by reference. Vaccine formulations according to the present invention are particularly suitable for species in the Salmo and Salvelinus genera, such as Atlantic salmon and brook trout.

The amount of attenuated Flavobacterium psychrophilum delivered to the fish is an amount that is effective to provide protection against disease caused by the non-attenuated bacteria. For example, if attenuated Flavobacterium psychrophilum is administered by immersion, the amount of bacteria in the water in which the fish are immersed is effective to provide protection, such as greater than 1×10⁴ CFU/ml, typically greater than 1×10⁵ CFU/ml, preferably greater than 1×10 6 CFU/ml, and may be as high as 1×10⁷ CFU/ml to 1×10¹⁰ CFU/ml. The water in which the fish are immersed may be fresh water, salt-water, or brackish, depending on the variety of fish to be treated and the natural habitat of the fish.

For immersion administration, the fish are immersed in the water containing the attenuated live bacteria for a time sufficient to provide protection against disease caused by non-attenuated bacteria. Generally, immersion times between 15 seconds and several hours are suitable, such as between 1 minute and two hours, and typical immersion times are between 30 minutes and 1 hour.

Protection against disease occurs when complete or partial immunity against the disease has been obtained. Immunity is obtained in a population of treated fish when the number of infected fish or severity of disease is higher in fish that have not been treated in accordance with the invention compared to vaccinated fish. Preferably, vaccination in accordance with disclosed embodiments of the present method will result in at least a 10% decrease in mortality, preferably a greater than 20% decrease in mortality to a 100% decrease in mortality, such as a 30% decrease, a 40% decrease, a 50% decrease, a 60% decrease, a 70% decrease, an 80% decrease, or a 90% decrease, compared to unvaccinated fish.

Diseases of fish that may be protected against by disclosed embodiments include any disease that is caused by a bacterium. In a preferred embodiment, the disease is Coldwater Disease (CWD), which is caused by Flavobacterium psychrophilum, and even more particularly is CWD in fish that are not effectively treated with prior vaccines, including the B17-ILM attenuated vaccine, such as fish species in the Salmo and Salvelinus genera. However, a person of ordinary skill in the art will appreciate that the method of the present invention concerning selecting a parent strain of an infectious bacteria having a sufficiently high virulence prior to attenuation to provide an attenuated strain that is effective in a vaccine can be applied to other diseases too, including both gram negative and gram positive bacteria, with one example including bacterial kidney disease caused by Renibacterium salmoninarum. In this specification, Flavobacterium psychrophilum and CWD are utilized as illustrative examples of bacteria and diseases.

Bacteria, such as Flavobacterium psychrophilum, are attenuated using any method by which the virulence of the bacteria may be reduced or eliminated. For example, the bacteria may be attenuated by exposing a wild-type strain of the bacteria to radiation or to a chemical compound that promotes mutations. Antibiotics, such as rifampicin, have been used to develop attenuated strains of the Flavobacterium psychrophilum strains identified herein. Attenuated bacteria are created by serial passaging wild-type or incompletely attenuated bacteria on or in media containing increasing rifampicin concentrations. Generally, increasing concentrations of rifampicin are utilized between 1μg rifampicin/ml up to the maximum amount of rifampicin soluble in TYES media, which is about 320 μg rifampicin/ml. Attenuation is generally most effective when final rifampicin concentrations are between about 200 rifampicin/ml and 280 μg rifampicin/ml, with certain embodiments using a final concentration of 260 μg rifampicin/ml. Passaging is continued for a suitable number of passages, generally at least about 10 passages. The Fp063-np13 strain has been completely attenuated by passaging at least 13 times to 260 ug/ml rifampicin.

Preferably, attenuated bacteria are completely attenuated and are no longer capable of producing disease in fish. For example, the Fp063-np13 strain has been completely attenuated by passaging at least 13 times to a final rifampicin concentration of 260 ug/ml or higher. However, there may be circumstances where it is advantageous to vaccinate fish with an incompletely attenuated live bacterial vaccine.

Biochemical and physical characteristics of the attenuated bacteria typically are not identical to those of the parent organism. Preferably, but not necessarily, there will be at least one difference in protein expression between the parent and attenuated bacteria. Such differences may be determined, for example, by changes in banding patterns such as on SDS PAGE and/or by Western blot. The attenuated bacteria may not express proteins that are expressed by the unattenuated bacteria, or may have reduced expression of such proteins. Alternatively, or in addition, the attenuated bacteria may express proteins that are not expressed in the unattenuated parent bacteria, or may express proteins to a greater extent than the unattenuated parent bacteria.

Similarly, production of lipopolysaccharides and/or carbohydrates may be different between parent and attenuated bacterial strains.

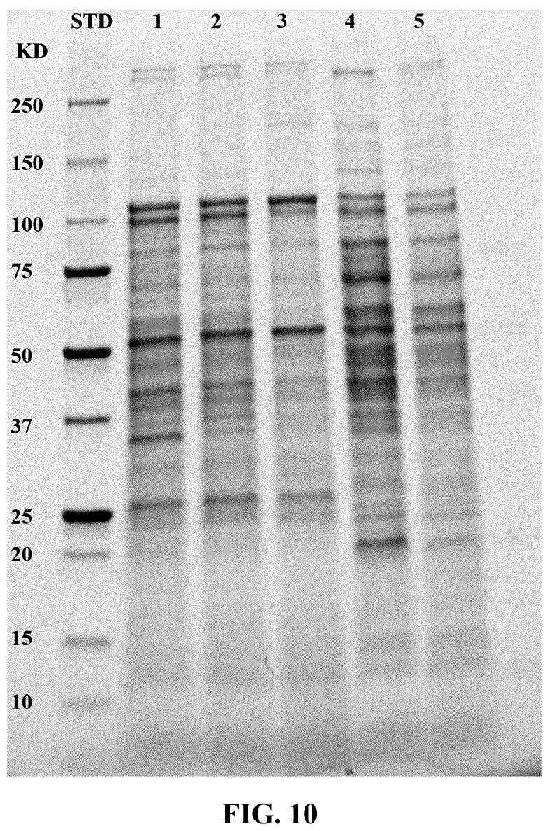

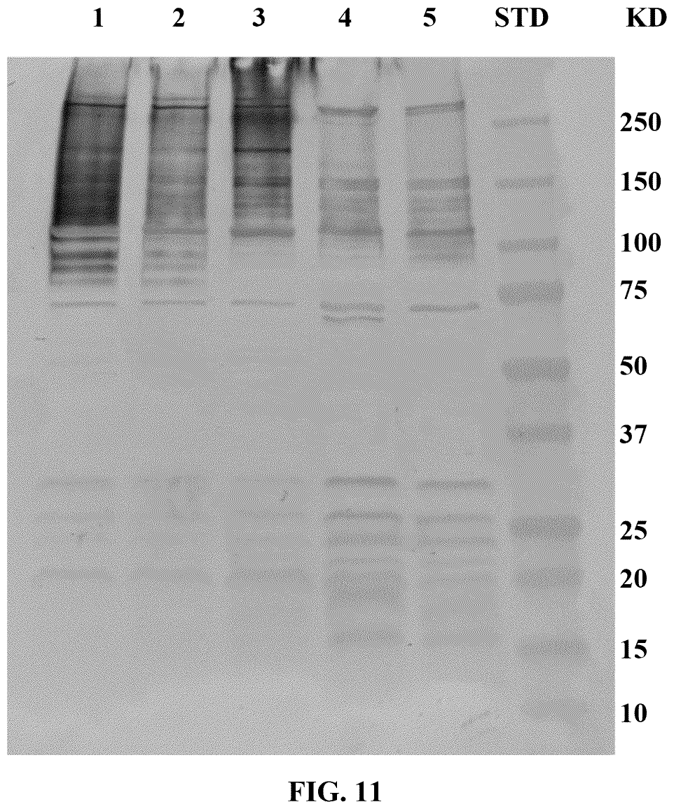

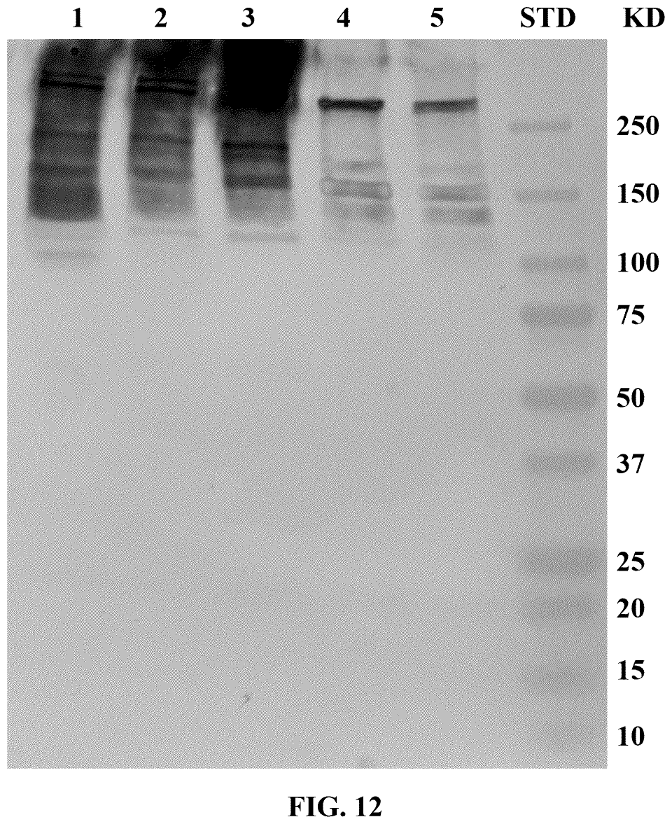

FIG. 10 is an image of an SDS-PAGE (sodium dodecyl sulfate-polyacrylamide electrophoresis gel comparing the protein profiles for an Fp063-np13 strain grown on an iron-limited medium (lane 1 — growth on iron-limited media is discussed in more detail by Example 4), Flavobacterium psychrophilum Fp063-np13 strain attenuated on TYES medium (lane 2), the US063 parent strain (lane 3), an attenuated Flavobacterium psychrophilum for the B.17 vaccine (lane 4), and Flavobacterium psychrophilum strain CSF259-93 (lane 5). The electrophoresis gel clearly establishes that there are substantial protein differences between each of the strains. For example, lane 2 provides a protein profile for an attenuated Flavobacterium psychrophilum strain according to the present invention. This protein profile can be compared to that of lane 4, an attenuated Flavobacterium psychrophilum for the known B.17 vaccine. FIGS. 11 and 12 are Western blots made using Atlantic salmon serum and Rainbow trout serum, respectively.

The SDS-PAGE protein profiles and Western blots are clearly distinct for each of the strains tested. The information provided by the SDS-PAGE protein profiles and Western blots establish that it not a simple, routine matter to identify an attenuated Flavobacterium psychrophilum strain different from that of the B.17 vaccine that would be efficacious for, for example, protecting Atlantic salmon against CWD since the protein profiles for each strain may be substantially distinct.

IV. Examples

The following examples are provided to illustrate certain features of the presently disclosed embodiments. A person of ordinary skill in the art will appreciate that the scope of the invention is not limited to such features.

### Example 1

A. Generation of Rifampicin Resistant Strains

Two virulent F. psychrophilum strains (US149 and US063) were used as the parent strains to generate rifampicin resistant strains. Previously frozen glycerol stocks of the US149 and US063 strains were plated separately for isolation on tryptone yeast extract salts (TYES; 0.4% tryptone, 0.04% yeast extract, 0.05% MgSO4·7H₂O, 0.05% CaCl₂·2H₂O, pH 7.2) agar and incubated at 15° C. for 5 days. A single colony was passed to TYES agar containing 2 μg/ml rifampicin (Sigma, St. Louis, MO, USA) and incubated at 15° C. for 6 days. For each strain, 3 of the resulting colonies were randomly selected, designated 149-mp17, 149-Anp16 and 149-np14 or 063-mp17, 063-Anp15 and Fp063-np13 (the number means passage number; p means passage; m or n means different initial colony/strain). The single colonies were then passed independently to TYES agar containing increasing concentrations of rifampicin. This process was repeated until the selected strains achieved growth at rifampicin concentrations of 260 μg/ml. Following initial virulence analysis, the Fp063-np13 strain was passaged two more times on TYES out to 280 μg/ml rifampicin. Following each passage, a portion of the growth was harvested, resuspended in sterile 20% glycerol, and frozen at −80° C.
B. Bacterial Culture

Growth curves of the US063 and Fp063-np13 strains were determined. Each strain was pre-cultured in 20 ml TYES broth at 15° C. for 72 hours, then was adjusted to at OD 525 nm with TYES. Triplicate culture tubes for each strain were inoculated with 50 μl of the adjusted cultures in 20 ml fresh TYES broth. All tubes were incubated at 15° C. for 8 days on an orbital shaker (100 rpm). Growth was monitored daily by measuring the optical densities at 525 nm.

For challenge trials, 100 μl of frozen glycerol stocks of each respective strain were inoculated into 20 ml TYES broth and cultured for 72 hours at 15° C.
C. Assessment of Virulence

Two experimental F. psychrophilum challenges were performed to assess the virulence of the parent and rifampicin-resistant strains. In trial 1, triplicate groups of 25 Atlantic salmon (mean weight 9.47 g/f) were challenged by intramuscular injection (IM) with 50 μl of the parent US149 or US063 strains, or selected strain 149-mp17, 149-Anp16 and 149-np14 or 063-mp17, 063-Anp15 and Fp063-np13 in TYES to optical densities (OD) of 0.4 at 525 nm (see Table below). A group of mock challenged controls (n=25) was injected with 50 μl of TYES.

In trial 2, quadruplicate groups of 25 Atlantic salmon (mean weight 14.2 g/f) were challenged by intraperitoneal injection (IP) of 50 ill of the parent US149 or US063 strain. Seven groups of 25 fish were IP injected with 50 ill of Fp063-np13 (one group for sampling and six groups for the immunization trial (assuming attenuation was observed). All the F. psychrophilum strains were adjusted to an OD of 0.4 at 525 nm (see Table below) in TYES. For each treatment, three groups were recorded for CPM and one group was used for tissue and blood sampling. A group of mock challenged controls (n=25) was injected with 50 μl of TYES to serve as controls. Mortalities were recorded daily for 28 days and re-isolation of F. psychrophilum was attempted on a minimum of 20% of the daily mortalities by inoculating spleen and kidney tissue onto TYES and TYES-R (TYES with rifampicin concentrations of 201.tg/ml) agar. The plates were incubated at 15° C. for 7 days, examined for yellow-pigmented bacteria phenotypic of F. psychrophilum and recorded as positive or negative. The CPM was calculated for each strain at the challenge doses tested.
D. Immunization Trial

Immunization trials were implemented to determine if Atlantic salmon injected with live Fp063-np13 attenuated strain would elicit a protective immune response against F. psychrophilum. Briefly, all Fp063-np13 IP injected (immunized) fish (mean weight 16.4 g/f) from virulence study trial 2 were moved to a single 100-liter tank. Following this, triplicate groups of 25 fish were challenged with 50 μl/f of virulent US149 or US063 strains (OD₅₂₅=0.15) by IM injection. Triplicate groups of 25 naïve unimmunized fish (mean weight 18.7g) from original stocks were challenged with the same dose of virulent US149 or US063. For the immunized or naïve fish, the mock-challenged controls were injected with 50 μl TYES. The CPM was determined for the treatment and control groups, and relative percent survival (RPS) of the treatment group was calculated as follows:

RPS=[1−(%mortality of vaccinated fish/%mortality of nonvaccinated fish)]×100
E. ELISA Analyses

Serum samples were obtained from Atlantic salmon in all treatments at the final day of virulence trial 1, and at 14- and 28-days post-challenge for virulence trial 2. Specific antibody titers against F. psychrophilum in serum samples were determined by an ELISA assay. Briefly, 100 HI, of F. psychrophilum US063 antigen were diluted in carbonate buffer to 10 μg/mL to all wells of each 96-well Immulon II plates, and the plates were then incubated at 4° C. overnight. After washing and blocking, the fish serum samples were serially diluted in doubling dilutions from 1:50 to 1:102400 in potassium phosphate buffered saline (KPBS)+0.05% (v/v) Tween-20 (KPBS-T) containing 0.02% sodium azide and incubated overnight at 15° C. Following washing, 100 μL of mouse monoclonal antibody against Salmonid immunoglobulin (Ig) (MCA2182, Bio-Rad Laboratories, Inc. CA) were added (1:500 in KPBS-T containing 0.1% non-fat dry milk) and incubated for 1 hour at room temperature. After washing, the plates were incubated for 1 hour at room temperature with 100 HI, horseradish peroxidase (HRP) conjugated goat anti-mouse Ig diluted 1:3000 in KPBS-T containing 0.1% non-fat dry milk (Bio-Rad Laboratories, Inc. CA). Plates were washed again, and bound enzyme was visualized using ABTS 2-Component Microwell Peroxidase Substrate Kit reagent A with 5.5 ml of reagent B (SeraCare Life Sciences, 5120-0032). After 15 minutes at room temperature, the color reaction was stopped by adding 50 μL of distilled water containing 1% sodium dodecyl sulphate. The OD of the wells was determined using a Bio-Tek Model EL 312E microplate autoreader at 405 nm (Bio-Tek Instruments Inc., Winooski, VT, USA). The ELISA titer was defined as the reciprocal of the highest dilution showing an OD at least two times greater than the negative control.
F. Bacterial Selection and Growth

For each strain, three resulting colonies were randomly selected, designated 149-mp17, 149-Anp16 and 149-np14 or 063-mp17, 063-Anp15 and Fp063-np13. All six colonies were passed independently to TYES agar containing increasing concentrations of rifampicin until the selected strains achieved growth at rifampicin concentrations of 260 μg/ml (FIG. 1).

Growth curves of the parent F. psychrophilum US063 and selected Fp063-np13 strains were determined in TYES broth at 15° C. (FIG. 2). Overall, the lag and exponential growth phases were similar for both strains from 0 to 8 days post-inoculation. For the first three days, the Fp063-np13 grew at a slower rate than the US063 and the final cell densities were lower, but without significant different.
G. Assessment of Virulence

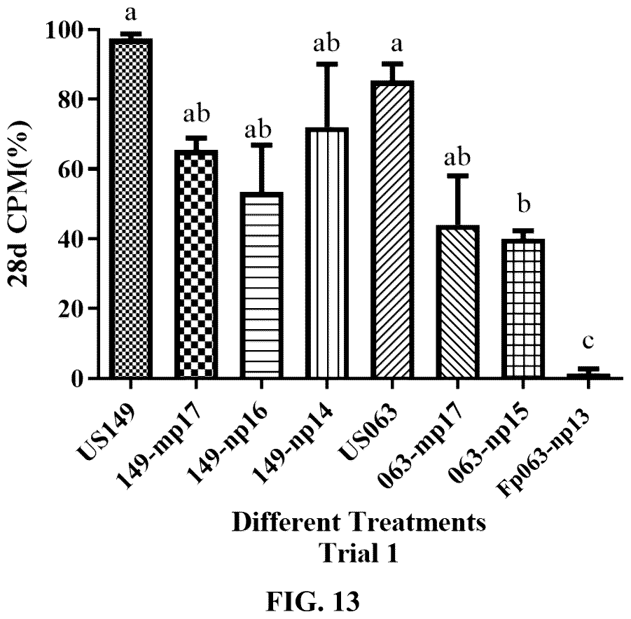

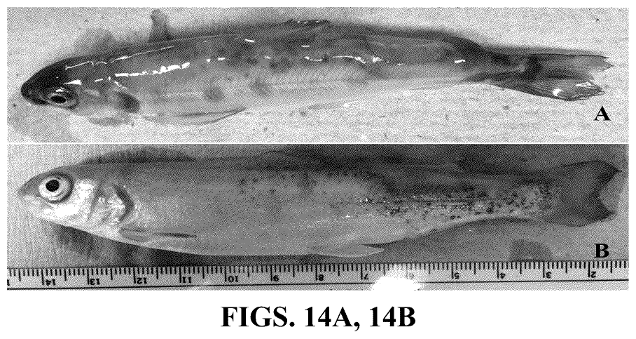

Atlantic salmon were challenged with parent strains and rifampicin-resistant strains to assess virulence. The results demonstrated almost complete attenuation of the Fp063-np13 strain and partial attenuation of the other 5 strains. In trial 1 (FIG. 13 and Table 4), the Fp063-np13 strain appeared highly attenuated with 1.33% CPM (one mortality in all three replicates (25 fish/tank) over the 28 days. Upon further analysis, this one mortality occurred on day four post injection and exhibited no clinical signs of disease, and no bacterial colonies were isolated on the TYSR-R plate from that fish. Some white bacterial colonies were isolated on the TYES plate. This indicates that this fish did not die as a result of the Fp063-np13 injection. The CPM of fish IM challenged in trial 1 with parent strains US149 or US063 was extremely high at 97.33%±0.02 and 85.33%±0.07, respectively. The three rifampicin-resistant strains derived from US149 caused 53.33% -88% CPM, while the other two rifampicin-resistant strains derived from US063 caused 40% or 44% CPM. Observed gross disease signs in challenged groups included extensive haemorrhagic or deep ulcerations on the dorsum (FIG. 14).

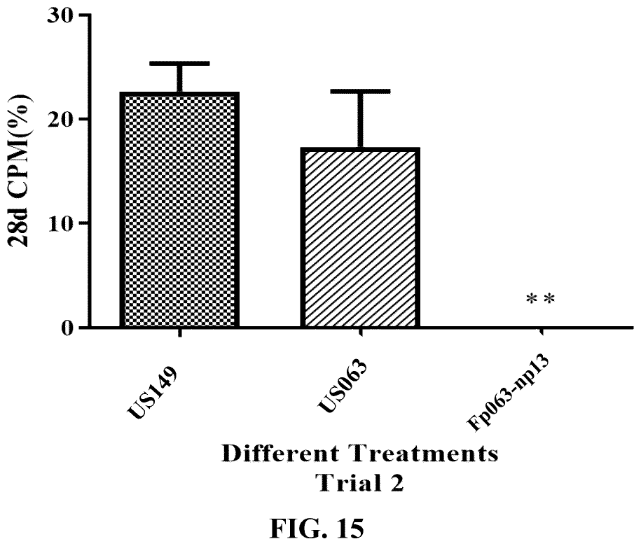

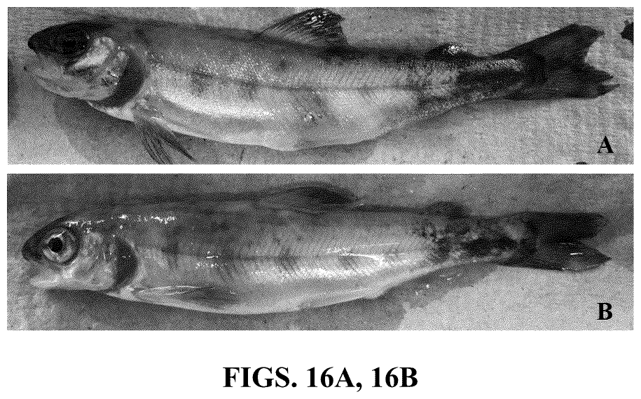

In trial 2, fish were nearly double the size as in trial 1 and parent strains US149 and US063 showed lower virulence (CPM 22.67% and 17.33%) by IP injection than by IM injection in trial 1, while the rifampicin-resistant strain Fp063-np13 exhibited complete attenuation and 100% survival for challenged fish (FIG. 15 and Table 5). The clinical signs in IP-challenged fish were different compared to the IM challenged fish, exhibiting unilateral exophthalmia and multifocal subcutaneous hemorrhage (FIG. 16).

TABLE 4
Calculated Dose of Flavobacterium Psychrophilum
Strains used in the Virulence Study with Intramuscular
Injection and 28 Days Post Injection CPM

| STRAINS | CFU/ML | CFU/FISH | 28 DPI CPM |
|---|---|---|---|
| US149 | 3.6 × 10⁹ | 1.8 × 10⁸ | 97.33% ± 0.02 |
| 149-mp17 | 1.07 × 10⁹ | 5.35 × 10⁷ | 65.33% ± 0.05 |
| 149--np16 | 1.1 × 10⁹ | 5.5 × 10⁸ | 53.33% ± 0.19 |
| 149-np14 | 1.01 × 10⁹ | 5.05 × 10⁸ | 88.00% ± 0.03 |
| US063 | 4.5 × 10⁹ | 2.25 × 10⁸ | 85.33% ± 0.07 |
| 063-mp17 | 1.02 × 10⁹ | 5.1 × 10⁸ | 44.00% ± 0.20 |
| 063-np15 | 8.0 × 10⁸ | 4 × 10⁸ | 40.00% ± 0.03 |
| Fp063-np13 | 2.9 × 10⁹ | 1.45 × 10⁸ | 1.33% ± 0.02 |

TABLE 5
Calculated Dose of F. Psychrophilum Strains used in the Virulence
Study with Intraperitoneal Injection and 28 Days Post Injection CPM

| STRAINS | CFU/ML | CFU/FISH | CPM |
|---|---|---|---|
| US149 | 2.28 × 10⁹ | 1.14 × 10⁸ | 22.67% ± 0.04 |
| US063 | 2.11 × 10⁹ | 1.06 × 10⁸ | 17.33% ± 0.08 |
| Fp063-np13 | 1.91 × 10⁹ | 9.55 × 10⁷ | 0 |

H. ELISA

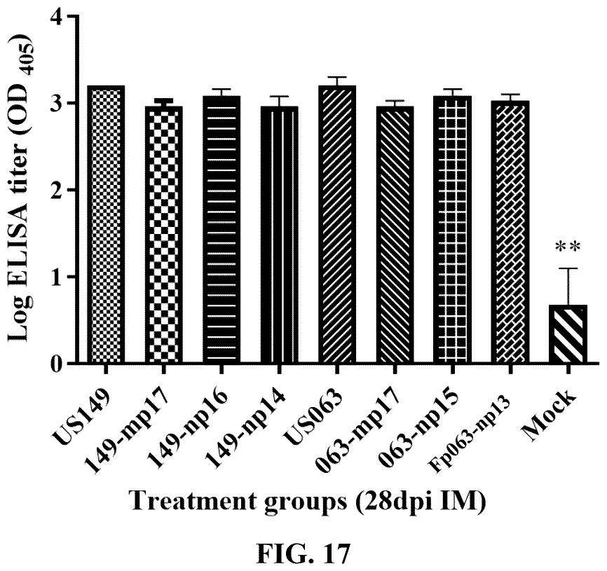

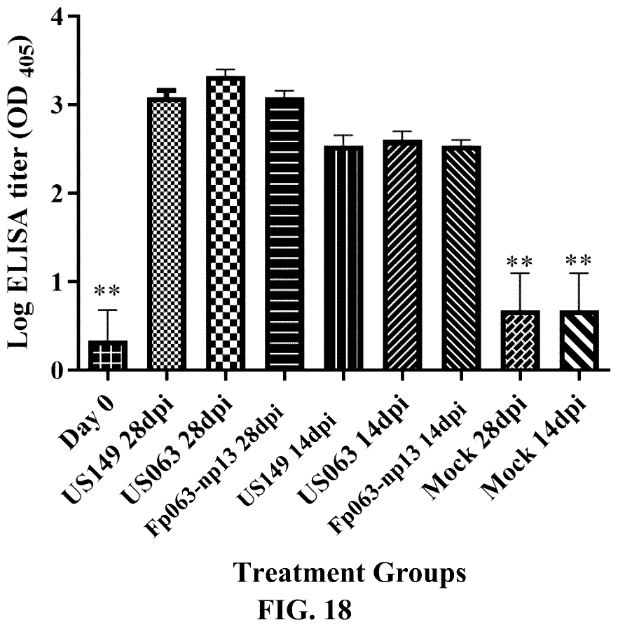

Initial screening of the Atlantic salmon for previous exposure to F. psychrophilum by an ELISA assay from five fish did not show any detectable anti-F. psychrophilum antibodies in serum. The immune response in Fp063-np13 IP-injected fish was detected at two weeks post injection. Surviving fish (28 days post challenge) from virulence studies trial 1 and trial 2 had elevated specific anti-F. psychrophilum antibody titers (FIGS. 17 and 18).

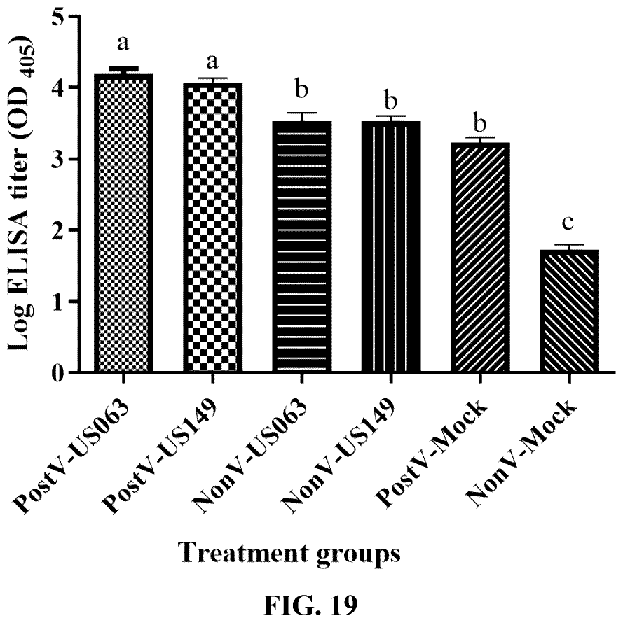

28 days post US149 or US063 challenge, the antibody titers in Fp063-np13-vaccinated fish were higher than the non-vaccinated fish (FIG. 19). The duration of antibody response (total 8 week post vaccinated) was seen in the vaccinated fish with mock challenge (3.23±0.16), which was higher than 28 days post vaccinated (3.08±0.16), but not significantly different.
I. Immunization Trial

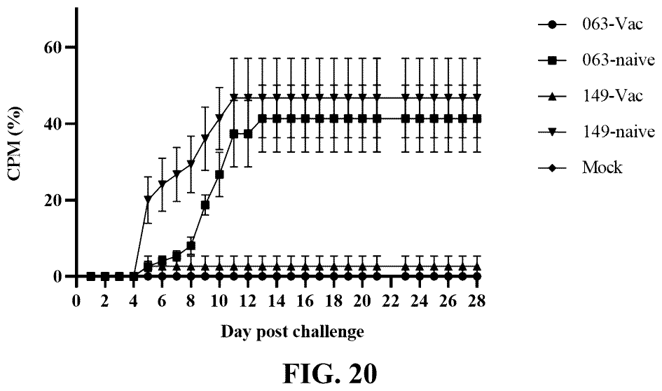

A protective immune response against F. psychrophilum was conferred to Atlantic salmon following immunization by IP injection with the Fp063-np13 strain (Table 6). Fish immunized with the Fp063-np13 strain exhibited a significantly decreased CPM, with 0% mortality in US063 challenged group and 2.67% (2/75) mortality in US149 challenged group. Conversely, naïve fish sustained high mortality, specifically with 46.67%±0.15 mortality in the US149 challenged group, and 41.33% ±mortality in the US063 challenged group at 28-day post challenge (FIG. 20). Relative percent survival values were 94.29%-100% (Table 6). There were no mortalities in the mock infected control groups. Challenge mortalities exhibited typical signs of F. psychrophilum infection. Yellow-pigmented bacteria phenotypically characteristic of F. psychrophilum were re-isolated from all mortalities.

TABLE 6
Calculated Dose of Virulent F. Psychrophilum Strains used in
the Immunization Study with Intramuscular Injection and CPM

| | | | 28 D CPM OF | CPM* OF | |
|---|---|---|---|---|---|
| | | | IMMUNIZED | NAÏVE | |
| STRAINS | CFU/ML | CFU/FISH | FISH | FISH | RPS |
| US149 | 7.17 × | 3.59 × | 2.67% ± | 46.67% ± | 94.29% |
| | 10⁸ | 10⁷ | 0.04 | 0.15 | |
| US063 | 3.2 × | 1.6 × | 0 | 41.33% ± | 100% |
| | 10⁸ | 10⁷ | | 0.12 | |

### Example 2

This example concerns immersion vaccination trials for Atlantic salmon.
A. Vaccination

Atlantic salmon (1.0 g/f) were vaccinated by immersion using a Fp063-np13 vaccine. Fp063-np13 vaccine bottles (previously stored at −80° C.) were thawed at 15° C. overnight and diluted 1:10, 1:100, or 1:1000 separately, in a total volume of 10 liters of clean rearing water immediately before vaccinating. 200 fish for each treatment were immersion vaccinated for 30 minutes. All vaccinated fish were booster vaccinated two weeks after primary vaccination using the same dose and methodology. Control groups were immersed in a 1:10 dilution of TYES media in an identical manner as the vaccinated fish.
B. Challenge:

After 8 weeks vaccination, triplicate groups of 25 fish (mean weight 6.5 g/f) were challenged with 50 ill of virulent US063 by IM injection. The CPM and RPS were determined for the treatment and control groups at each treatment.

TABLE 7
Calculated dose of F. psychrophilum Live Attenuated
and Virulent Strains by Spread Plate

| STRAINS | CFU/ML | OD₅₂₅ |
|---|---|---|
| Fp063-np13 | 1.33 × 10¹⁰ | 0.51 |
| US063 | 1.42 × 10⁸ | 0.071 |

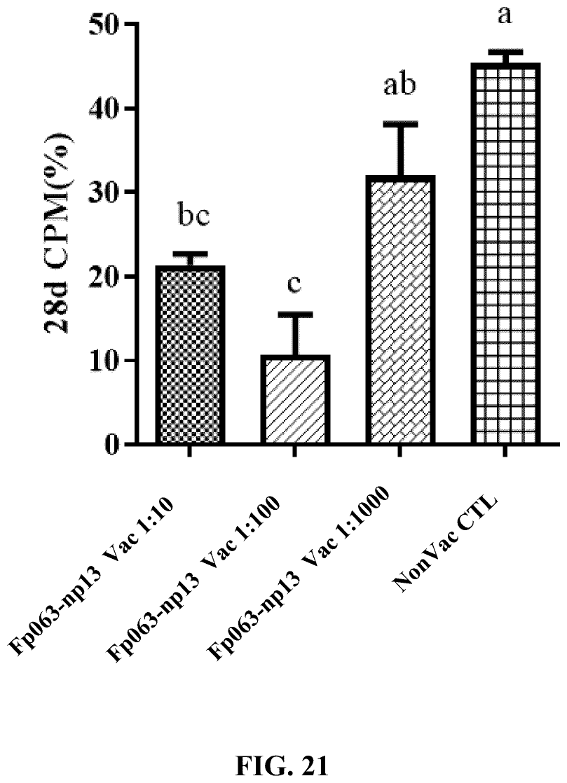

The 28 days CPM for fish challenged with the US063 strain was the highest with (non-vaccinated group), while the vaccinated groups had low CPMs. The 1:100 dilution group had the lowest CPM (10.67%) and highest RPS (76.47%). See Table 8 and FIG. 21.

TABLE 8
Cumulative Percent Mortality and Relative Percent
Survival of Atlantic Salmon 28 Days Post Challenge

| | CPM | RPS |
|---|---|---|
| Non Vac Control | 45.33% ± 0.024 | |
| Fp063-np13 Vac 1:1000 | 32.00% ± 0.106 | 29.41% |
| Fp063-np13 Vac 1:100 | 10.67% ± 0.083 | 76.47% |
| Fp063-np13 Vac 1:10 | 21.33% ± 0.023 | 52.90% |

C. ELISA

Initial screening of the Atlantic Salmon for previous exposure to F. psychrophilum by an ELISA assay from 10 fish did not show any detectable anti-F. psychrophilum antibodies in serum.

The immune responses in Fp063-np13 immersed fish were detected with an elevated specific anti-F. psychrophilum antibody titer after 2 weeks post vaccination. From 6 weeks post vaccination, the vaccinated fish had significant higher F.

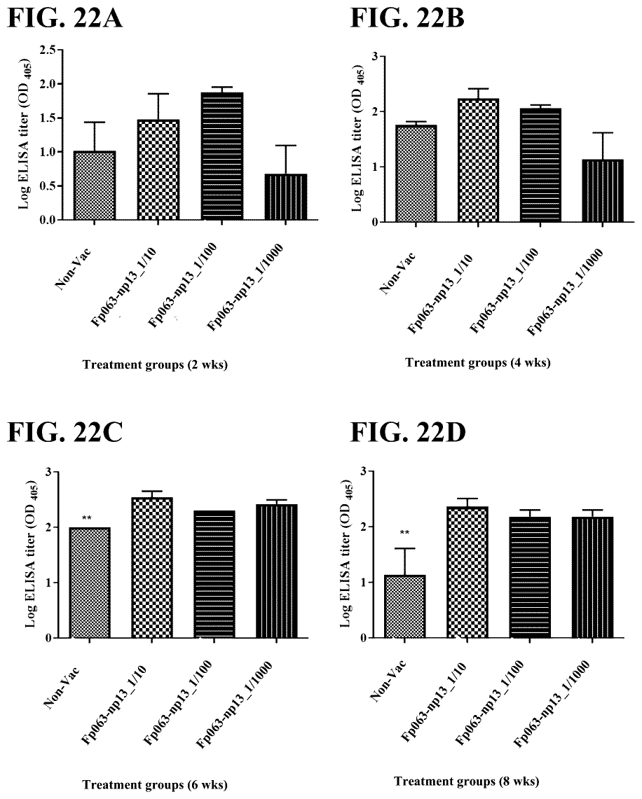

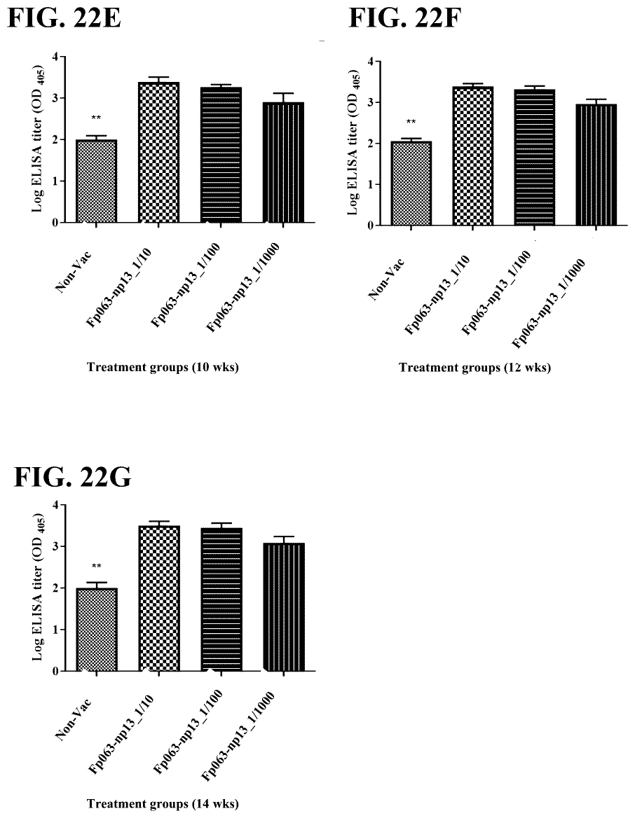

psychrophilum antibody titers than non-vaccinated. At 14 weeks post vaccination, the 1/10 dilution group had the highest titer, but without significant difference with other dilution groups. See FIGS. 22A-22G.

### Example 3

This example concerns immersion vaccination trials for Rainbow trout.
A. Vaccination

180 Rainbow trout (1.46g/f) were vaccinated by standard immersion methods using a Fp063-np13 vaccine. For the immersion vaccination, Fp063-np13 vaccine bottles (previously stored at -80° C.) were thawed at 15° C. overnight and diluted 1:10 in a total volume of 10 liters of clean rearing water immediately before vaccinating. Fish were immersion vaccinated for 30 minutes and all vaccinated fish were booster vaccinated two weeks after receiving the primary vaccination using the same immersion methodology. Control groups were immersed in a 1:10 dilution of TYES media in an identical manner as the vaccinated fish.
B. Challenge

Eight weeks post vaccination, triplicate groups of 23 fish (mean weight 6.33g/f) were challenged with 50 ill of virulent US063 or CSF 259-93 by IM injection (Table 9). CPM and RPS were determined for the treatment and control groups at each treatment.

TABLE 9
Calculated Dose of F. Psychrophilum Live Attenuated
and Virulent Strains by Spread Plate

| STRAINS | CFU/ML | OD₅₂₅ |
|---|---|---|
| Fp063-np13 | 1.33 × 10¹⁰ | 0.51 |
| US063 | 1.32 × 10⁸ | 0.12 |
| CSF 259-93 | 5.40 × 10⁷ | 0.12 |

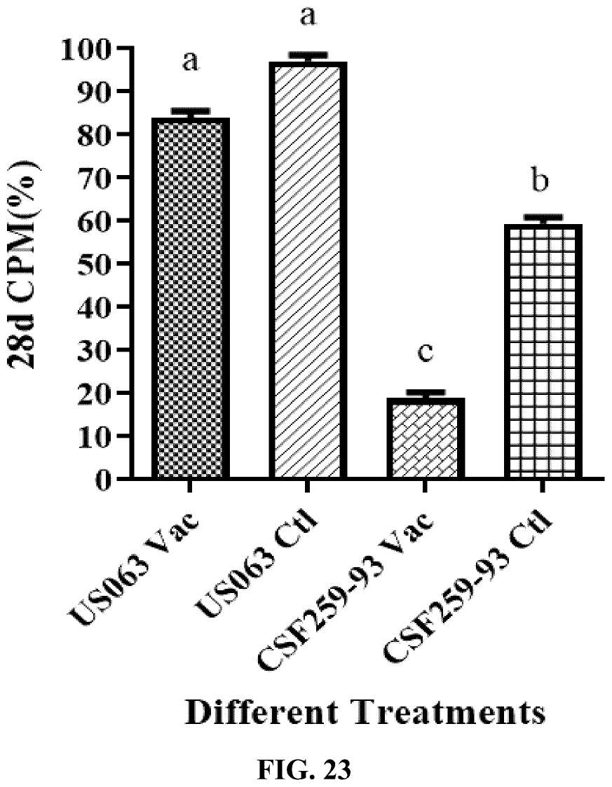

The CPM of fish challenged with the US063 strain was extremely high with 97.10% (non-vaccinated group) or 84.06% (vaccinated group), indicating the potential protection was overwhelmed by the high virulence or dose. 28 days after CSF 259-93 challenge, the non-vaccinated group had a 59.42% CPM, while the vaccinated group had an 18.84% CPM and the RPS was 68.29%. See FIG. 23.
C. ELISA

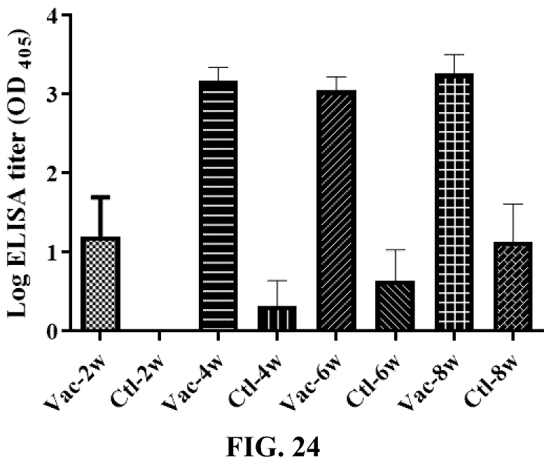

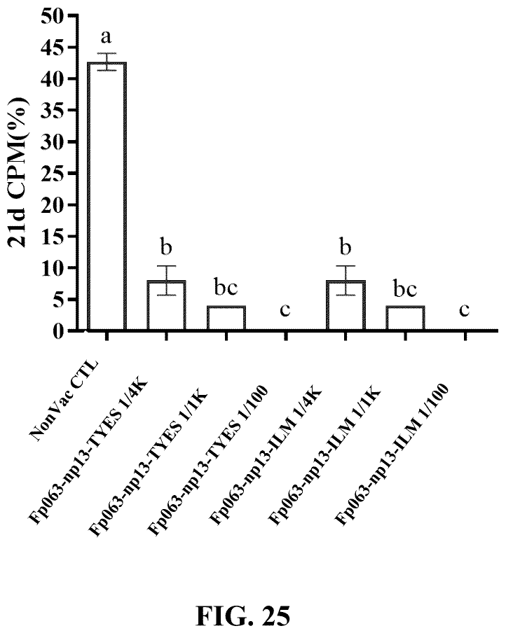

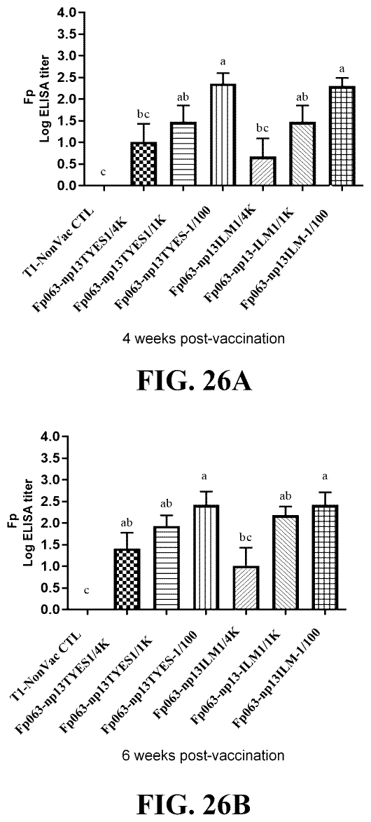

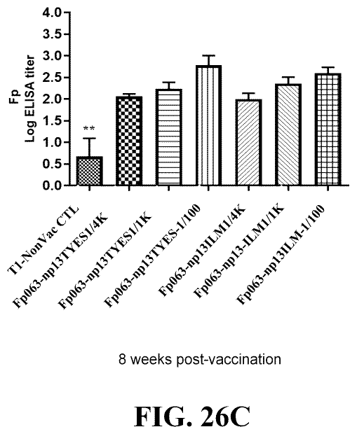

Initial screening of the rainbow trout for previous exposure to F. psychrophilum by an ELISA assay from five fish did not show any detectable anti-F. psychrophilum antibodies in serum. The immune response in Fp063-np13 immersed fish was detected with an elevated specific anti-F. psychrophilum antibody titer after 2 weeks post vaccination. See FIG. 24.

### Example 4

This example concerns vaccination of rainbow trout by immersion with an Fp063-np13-TYES vaccine or an Fp063-np13-ILM vaccine. The term “iron-limited” means a medium that is essentially free of iron or, if iron is present in the medium, then the medium also contains an iron chelator at a concentration to sufficiently to reduce the amount of free iron in the medium. A live attenuated strain of the bacterium is grown in or on an iron-limited medium and the bacterium is then harvested. Growing attenuated bacterium strains in or on an iron-limited medium has been shown for certain fish vaccines to increase the efficacy of such vaccines to reduce morbidity and/or mortality compared to a similar vaccine comprising the same bacterium strain but grown in or on a medium that is not iron-limited.

In order to test this conception, an attenuated strain of a bacterium known to cause disease in fish may be grown (a) in an iron-limited medium, or (b) in an iron-rich medium. Fish arethen vaccinated with the strain grown in one of these two media, and the levels of protection due to the two vaccination protocols are compared. Protection is determined as percentage survival following challenge.

The medium in or on which the attenuated strain is grown is any medium that will support growth of the bacterial strain. For example, tryptone yeast extract salt (TYES) in liquid or solid form may be utilized. Other examples include tryptic soy liquid or solid medium (TSB or TSA), brain heart infusion agar or broth, Cytophaga agar or broth, Anacker and Ordal liquid or solid medium, Hsu-Shotts liquid or solid medium, MAT liquid or solid medium, and Shieh liquid or solid medium.
A. Bacterial Culture

Fp063-np13 was stored at — 80° C. as a master seed stock in TYES broth medium with 20% glycerol. A cryo-tube from the master seed stock was withdrawn, thawed and 200 μL of this stock culture was used to inoculate 10 mL of TYES medium and incubated at 15° C. for 72 hours with shaking at 80 rpm. To grow the strain under iron limiting conditions (ILM), named as Fp063-np13-ILM, the whole 10 mL culture was inoculated to 250 mL TYES medium containing the iron chelator, 2, 2-dipyridyl (DPD) (Sigma Aldrich, St. Louis, MO, USA) at a final concentration of 50 μM. The culture was allowed to grow at 15° C. for 72 hours with shaking at 120 rpm. The culture was scaled up to 1 L and then to 4 L volumes in TYES containing 50 μM DPD and incubated at 15° C. for 96 hours with shaking at 150 rpm. This final vaccine culture broth was mixed with sterile glycerol to a final concentration of 15% glycerol, mixed well and aseptically poured into sterile 1 L serum bottles (Genesis Industries Inc, WI, USA), closed with sterile rubber stopper and sealed with aluminum seals using a crimper. All bottles were labeled and stored at −80° C. until vaccinated. The vaccine that was cultured in the iron-replete medium (named as Fp063-np13-TYES) for all the steps was used to evaluate the results obtained using the Fp063-np13-ILM vaccine.
B. Rainbow Trout Vaccination by Immersion with the Fp063-Np13-TYES Vaccine or the Fp063-Np13-ILM Vaccine

Approximately 1,750 rainbow trout were distributed evenly into 7 circular tanks, 250 fish for each tank. Table 10 below shows the design and treatment Groups for this study:

TABLE 10
Design and Treatment Groups

| GROUPS | VACCINE | DOSE/DILUTION |
|---|---|---|
| 1 | TYES (Non Vac Control) | 1:100 |
| 2 | Fp063-np13-TYES | 1:4000 |
| 3 | Fp063-np13-TYES | 1:1000 |
| 4 | Fp063-np13-TYES | 1:100 |
| 5 | Fp063-np13-ILM | 1:4000 |
| 6 | Fp063-np13-ILM | 1:1000 |
| 7 | Fp063-np13-ILM | 1:100 |

The vaccine or TYES was diluted in a total volume of 20 L using clean rearing water. The fish were immediately moved back to their rearing tanks after 30 minutes of immersion in the diluted vaccine or TYES. Two weeks following initial vaccination, all fish were booster vaccinated in an identical manner as primary vaccination. The Fp063-np13-TYES vaccine had an OD525 value of 0.80, corresponding to 4.2×10⁹ CFU/ml. The Fp063-np13-ILM vaccine had an OD525 value of 0.77, corresponding to 3.6×10⁹ CFU/ml.
C. Fish Challenge

8 weeks following initial vaccination, fish (average weight=11.3 g/f) from each treatment were divided into triplicate tanks (25 fish/tank) for challenge with 50 μl virulence F. psychrophilum US063 stain. The US063 culture for challenge had an OD525 value of 0.055, corresponding to 7.8×10⁷ CFU/ml (3.9×10⁶ CFU/fish).

The 21 days CPM for fish challenged with the US063 strain was highest for the non-vaccinated group at 42.7%, while the vaccinated groups had significantly lower CPMs. The 1:100 dilution treatment for both ILM and TYES groups had no mortalities, with a 100% RPS. For each vaccine dilution treatment (1/4000, 1/1000 or 1/100), the mean CPMs of fish immunized with vaccine cultured in TYES or ILM culture medium were the same throughout the experiment.

TABLE 11
Cumulative Percent Mortality and Relative Percent Survival
of Rainbow Trout at the End of the 21 Days of Challenge

| TREATMENT | CPM | RPS |
|---|---|---|
| TYES (NonVac Control) | 42.7% | NA |
| Fp063-np13-TYES 1/4K | 8.0% | 81.3% |
| Fp063-np13-TYES-1/1K | 4.0% | 90.6% |
| Fp063-np13-TYES-1/100 | 0.0% | 100.0% |
| Fp063-np13-ILM-1/4K | 8.0% | 81.3% |
| Fp063-np13-ILM-1/1K | 4.0% | 90.6% |
| Fp063-np13-ILM-1/100 | 0.0% | 100.0% |

D. ELISA

Initial screening of the rainbow trout for previous exposure to F. psychrophilum by an ELISA assay from 5 fish did not show any detectable anti-F. psychrophilum antibodies in serum. The immune response in Fp063-np13 immersed fish was detected with an elevated specific anti-F. psychrophilum antibody titer after 4 weeks post-vaccination.

The response in the vaccinated fish from all groups increased out to 8 weeks and was significantly higher than non-vaccinated control fish. The 1:100 dilution groups had the highest antibody response at all-time points, but without significant difference from 1:1000 dilutions groups.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. A bacterial cold-water disease (BCWD) vaccine for fish in the Salmonidae family comprising an attenuated Flavobacterium psychrophilum strain prepared from an unattenuated Flavobacterium psychrophilum parent strain having a 40% or greater cumulative percent mortality (CPM) in fish weighing from 5-10 grams that are challenged intramuscularly with the parent strain at a dose of at least 1×10⁷ cfu/fish.

2. The vaccine according to claim 1, wherein:
the unattenuated Flavobacterium psychrophilum parent strain was U5063;
the CPM in Atlantic salmon was 50% or greater; and
the CPM in brook trout was 60% or greater.

3. The vaccine according to claim 1, wherein:
the unattenuated Flavobacterium psychrophilum parent strain was US149;
the CPM in Atlantic salmon was 90% or greater; and
the CPM in brook trout was 40% or greater.

4. The bacterial cold-water disease (BCWD) vaccine according to claim 1 formulated for fish in the Salmonidae family, and comprising a live, attenuated Flavobacterium psychrophilum strain.

5. The bacterial cold-water disease (BCWD) vaccine for fish according to claim 4 formulated particularly for Atlantic salmon, brook trout, and rainbow trout comprising a live, attenuated Flavobacterium psychrophilum strain derived from US149, US063 (ST278), or both.

6. The vaccine according to claim 1 comprising two or more different live, attenuated Flavobacterium psychrophilum strains.

7. The vaccine according to claim 1 comprising 10 3 CFU/ml to 10 10 CFU/ml of the live attenuated Flavobacterium psychrophilum.

8. The vaccine according to claim 1 further comprising water, physiological saline, a mineral oil, a vegetable oil, aqueous sodium carboxymethyl cellulose, aqueous polyvinylpyrrolidone, an adjuvant, alum, Freund's incomplete adjuvant, and combinations thereof.

9. The vaccine according to claim 1 wherein the Flavobacterium psychrophilum is a completely attenuated Fp063-np13 strain.

10. A bacterial cold-water disease (BCWD) vaccine for fish species of the Salmo and Salvelinus genera comprising a live attenuated Flavobacterium psychrophilum strain prepared from an unattenuated Flavobacterium psychrophilum parent strain having a 40% or greater cumulative percent mortality (CPM) in fish weighing from 5-10 grams that are challenged intramuscularly with the parent strain at a dose of at least 1×10⁷ cfu/fish.

11. The vaccine according to claim 10 wherein the Flavobacterium psychrophilum strain results in a cumulative percent mortality (CPM) in the fish species challenged with the strain of 70% or greater without attenuation.

12. The bacterial cold-water disease (BCWD) vaccine according to claim 10, consisting of:
10⁴ CFU/ml to 10 9 CFU/ml of a live attenuated Flavobacterium psychrophilum strain prepared from an unattenuated parent strain designated US149, US063 (ST278), or both, the unattenuated strain having a 40% or greater cumulative percent mortality (CPM) in fish weighing from 5-10 grams that are challenged intramuscularly with the parent strain at a dose of at least 1×10⁷ cfu/fish; and
water, physiological saline, a mineral oil, a vegetable oil, aqueous sodium carboxymethyl cellulose, aqueous polyvinylpyrrolidone, an adjuvant, alum, Freund's incomplete adjuvant, and combinations thereof.

13. The vaccine according to claim 10 wherein the Flavobacterium psychrophilum strain is Fp063-np13.

14. A method, comprising:
providing a bacterial cold-water disease (BCWD) vaccine according to claim 1; and
administering at least a first dose comprising an effective amount of the vaccine to fish.

15. The method according to claim 14, wherein the fish species are of the Salmo and Salvelinus genera.

16. The method according to claim 14, wherein the vaccine comprises 10 4 CFU/ml to 10 9 CFU/ml of the live attenuated Flavobacterium psychrophilum.

17. The method according to claim 14, comprising immersing fish in a 1:10 to 1:10000 volume dilution of the vaccine for an effective period of time of at least 30 minutes.

18. The method according to claim 14 comprising administering an amount of the vaccine by injection to provide 10⁷ CFU/fish to10⁸ CFU/fish of the live attenuated Flavobacterium psychrophilum.

19. The method according to claim 14 comprising administering a booster to previously vaccinated fish.

20. The method according to claim 14 further comprising administering a second therapeutic to the fish species, wherein the second therapeutic is oxytetracycline, florfenicol, or combinations thereof.

21. A method for making a CWD vaccine, comprising:
identifying a highly virulent Flavobacterium psychrophilum bacterial strain for a target fish genus or species of interest;
attenuating the highly virulent Flavobacterium psychrophilum bacterial strain; and
producing a live, attenuated vaccine comprising, consisting of, or consisting essentially of, the attenuated highly virulent Flavobacterium psychrophilum bacterial strain.

22. The method according to claim 21, wherein the highly virulent Flavobacterium psychrophilum bacterial strain has a 40% or greater cumulative percent mortality (CPM) in fish species in the Salmonidae family weighing from 5-10 grams that are challenged intramuscularly with the parent strain at a dose of at least 1×10⁷ cfu/fish.

23. The method according to claim 21, wherein the highly virulent Flavobacterium psychrophilum bacterial strain is selected from US149 and US063 strains.

24. The method according to claim 21, wherein the highly virulent Flavobacterium psychrophilum bacterial strain is Fp063-np13.

25. The method according to claim 21, further comprising growing a live, attenuated highly virulent Flavobacterium psychrophilum bacterial strain on iron-limited media.
